(19) United States
(12) Patent Application Publication
(10) Pub. No.: US 2019/0192703 A1
(43) Pub. Date: Jun. 27, 2019

(54) TREATMENT OF NON-HODGKIN LYMPHOMA USING LILOTOMAB AND 177LU-LILOTOMAB SATETRAXETAN

(71) Applicant: Nordic Nanovector ASA, Oslo (NO)

(72) Inventors: Jostein Dahle, Haslum (NO); Simon Turner, Oslo (NO)

(21) Appl. No.: 16/329,661

(22) PCT Filed: Sep. 15, 2017
(86) PCT No.: PCT/EP2017/073336

§ 371 Date: Feb. 28, 2019

(30) Foreign Application Priority Data
Sep. 16, 2016 (EP) 16189191.6
Mar. 31, 2017 (EP) 17164164.0
May 11, 2017 (EP) 17170641.9
Jun. 13, 2017 (EP) 17175768.5

(51) Int. Cl. A61K 51/10; C07K 16/28; C07K 16/30; A61P 35/00

(52) U.S. Cl. CPC A61K 51/1096; A61K 51/1027; A61K 51/1069; A61K 2039/507; C07K 16/2896; C07K 16/3061; A61P 35/00; C07K 16/2887

(57) ABSTRACT

The disclosure relates to the use of ¹⁷⁷Lu-lilotomab satetraxetan in the treatment of Non-Hodgkin lymphoma. Aspects included are specific administration patterns, with specific concentrations, pre-treatments and predosing.

### FIELD OF THE INVENTION

The invention relates to the use of monoclonal antibodies conjugated with ¹⁷⁷Lu in the treatment of Non-Hodgkin lymphoma. Aspects included specific administration patterns, with specific concentrations, pre-treatments and predosing, wherein ¹⁷⁷Lu-lilotomab satetraxetan is the central medicament.

### BACKGROUND OF THE INVENTION

Non-Hodgkin Lymphomas (NHL) as a group is the most common malignant haematological disease. NHLs are a diverse group of blood cancers that include any kind of lymphoma except Hodgkin lymphoma. NHLs are tumours developed from lymphocytes, a type of white blood cells. NHLs vary in their clinical behaviour, morphologic appearance, immunologic and molecular phenotype. The various types represent neoplastic lymphoid cells arrested at different stages of differentiation. Based on their natural history, NHLs can be clinically classified as indolent, aggressive, and highly aggressive. Diffuse large B-cell and follicular lymphoma are the most common subtypes.

NHLs are the fifth most common cause of cancer in the United States, with an estimated incidence of 70,130 cases in 2012. Follicular center cell lymphomas are the second most common subtype, comprising approximately 40% of all NHLs. Since 1950, the incidence of NHL has steadily increased at approximately 4% per year. Treatment usually depends on the type of lymphoma and its stage, as well as other prognostic factors. The different treatment options are radiation therapy, chemotherapy, immunotherapy, radioimmunotherapy (RIT) and bone marrow or peripheral stem cell transplantation. In B-cell and follicular lymphoma, rituximab (immunotherapy) combined with chemotherapy or a combination of drugs such as CHOP (cyclophosphamide, hydroxydaunorubicin, oncovin and prednisone) regimen is used.

Patients with relapsed indolent lymphoma may repeatedly respond to rituximab, chemotherapy combined with rituximab or other chemotherapy combinations although the proportion responding decreases with each relapse.

The aim of RIT is to use a monoclonal antibody (MoAb) to target an isotope for radiation to tumour tissue while limiting the toxicity to normal cells. Beta-emitting radioimmunoconjugates (RIC) possess high levels of clinical activity in patients with relapsed or refractory B-cell lymphomas, including those refractory to rituximab and chemotherapy. Clinical data have validated that RIT is both more cost effective and more efficacious than nonradioactive immunotherapy. More recently, several single-arm studies have demonstrated that upfront RIT administered either alone or with chemotherapy to previously untreated indolent NHL patients produces overall response rates of 90-100%, complete response rates of 60-95% and durable remissions.

A phase III study of RIT as part of frontline therapy for indolent NHL reported that consolidation therapy with ⁹⁰Y-ibritumomab tiuxetan (Zevalin) after induction chemotherapy markedly prolonged progression free survival in patients with previously untreated stage II or IV follicular lymphoma. In another study, patients with indolent and aggressive NHLs received four cycles of chemotherapy followed by high myeloablative dose ⁹⁰Y-ibritumomab tiuxetan followed by autologous stem cell support.

After a follow up time of 30 months, the overall survival rate was 87% and the event free survival was 69%. Although myeloablative doses of ⁹⁰Y-ibritumomab tiuxetan were given, the RIT was well tolerated. The low dose-rate permits RIT to be effective for haematologic malignancies while causing minimal non-haematological toxicity.

When anti-CD20 RIT is given to patients, they are administered with large quantities of unlabeled cold anti-CD20 antibody immediately before radiolabelled anti-CD20 antibodies. Such a priming dose is necessary to optimize radiolabelled antibody concentrations in tumour, presumably by partially saturating easily accessible B-cells in the blood and the spleen and permitting sufficient radiolabelled antibody to bypass these sites and penetrate less accessible compartments such as lymph nodes and large tumour masses.

However, too much cold anti-CD20 antibody over a long time can result in blocking of the CD20 antigen on tumour cells and thus reduce the effect of anti-CD20 RIT. Both clinical and non-clinical studies have shown that in some circumstances quite low rituximab concentrations in the blood can reduce tumour cell targeting and thus impair the clinical efficacy of CD20-directed RIT. A solution to this problem might be to omit cold rituximab from the last cycles of therapy before RIT. Alternatively; one could choose to target another B-cell surface antigen such as CD37.

RIT with CD37 as the target antigen has been explored previously using a ¹³¹I-labeled murine monoclonal antibody (MB-1) both in a mouse model and in patients with low, intermediate and high-risk NHLs. CD37 antibodies were compared with CD20 antibodies and a higher grade of internalization and degradation of ¹³¹I-labeled RIC was found for CD37 than for CD20.

Furthermore, a favorable biodistribution was obtained in 59% of the patients for CD20 and for 50% of the patients for CD37. The amount of cold priming with antibody necessary to get a favorable biodistribution was higher for CD37 than for CD20. All six patients treated with ¹³¹I-MB-1 (against CD37 antibody) had a complete response and three of the patients received bone marrow transplantation. Of twelve patients that were treated with ¹³¹I labeled antibody against CD20 ten had a complete response and eleven needed bone marrow transplantation. Despite the clinical responses observed in this study, the data for CD20 was evaluated to be marginally better than for CD37.

CD20 was therefore chosen as the target antigen for further development of a commercially available radioimmunoconjugate (RIC). This development resulted in FDA approval of Bexxar and Zevalin in 2003. No subsequent efforts have been made to target CD37 with RICs. Trubion Pharmaceuticals have however developed a nonradioactive CD37-binding small modular immunopharmaceutical that induces apoptosis and antibody-dependent cellular cytotoxicity against B-cell leukemia/lymphoma cell lines and primary chronic lymphocytic leukaemia cells. Previous studies thus show that CD37 is a potent target for both immunotherapy and RIT.

The chloramine T method of ¹³¹I-labeling was used in the early studies of CD37 RIT described above. ¹³¹I labeled to antibodies with the iodogen or the chloramine T method are not being contained in the cells if the antigen-antibody complex is internalized. Inside the cells the nuclide is removed from the antibody by intracellular enzymes and diffuses out and away from the tumour cells. The same so-called dehalogenation has been shown with CD22 antibodies, which are also internalized. Metallic radionuclides labeled to antibodies with so-called chelators are however more stable and remain contained inside the cells to a much higher degree. By using metallic radionuclides internalizing antigens can be used for tumour targeting and tumour uptake may also be higher than for non-internalizing antibodies as well.

At the Norwegian Radium Hospital, an antibody (lilotomab also called HH1) was developed against CD37 in the 1980's. Lilotomab and the anti-CD20 antibody rituximab have been labeled with both ¹²⁵I and ¹¹¹In and measured cell bound activity after 4 days of incubation with a lymphoma cell line. The results show that the problem of catabolism of RIC can be circumvented by labeling with metallic nuclides such as ¹¹¹In or ¹⁷⁷Lu.

The most common radiopharmaceuticals used in therapy today utilize substances that disintegrate resulting in the emission of a beta particle. Beta particles are electrons emitted from the nucleus of an atom. Beta emitters approved for therapy include Iodine-131 (T½=8 days), Yttrium-90 (T½=2.7 days) and Lutetium-177 (T½=6.7 days). ¹⁷⁷Lu has been selected for use in Betalutin since it has proven to be suitable for labeling of the antibody and has an appropriate energy of the emitted 3-particle (Emax=0.497 MeV, T½=6.7 days). Furthermore, it has a low abundance of photons with almost ideal energy for imaging (E=113 keV, abundance=6.5%; E=208 keV, abundance=11%).

Betalutin (lilotomab labeled with ¹⁷⁷Lu via the chelator p-SCN-benzyl-DOTA, or ¹⁷⁷Lu-lilotomab satetraxetan) has been developed by Nordic Nanovector in collaboration with the Norwegian Radium Hospital for the treatment of relapsed NHL.

RIT permits delivery of a therapeutic dose of radiation directly to the DNA of tumour cells. The radionuclide ¹⁷⁷Lu is a beta-particle emitter. The beta particles are electrons with energy and range in tissue suitable for treating NHLs. The absorbed radiation results in DNA damage and tumour cell death. The radiation emitted from the radiolabeled antibody affects not only the antibody-binding cell, but also neighbouring cells. This mechanism of action of RIT may be especially beneficial in treating patients with bulky or poorly vascularized tumours.

Betalutin has been tested for targeting, therapeutic and toxic effect in cells and in mice. Lilotomab has similar or better binding properties to CD37 as rituximab has to CD20. Therapy against single cells showed a significantly better effect of Betalutin than of ¹⁷⁷Lu-rituximab. The MTD of Betalutin in SCID mice with tumour cells in the bone marrow was between 50 and 100 MBq/kg (Dahle et al. 2013). In studies with nude mice without tumour cells in the bone marrow the MTD is above 500 MBq/kg (Repetto et al. 2015).

Biodistribution studies with Betalutin have shown high uptake in tumour and uptake in normal organs similar to the uptake of ¹⁷⁷Lu-rituximab. The preclinical data to date indicate that Betalutin has a suitable biodistribution profile with high uptake in tumour cells, and that the efficacy results in the mouse models show promise of potentially interesting clinical results.

Thus, ¹⁷⁷Lu-lilotomab satetraxetan is a candidate for the treatment of Non-Hodgkin lymphoma.

However, there are serious challenges before ¹⁷⁷Lu-lilotomab satetraxetan can be used in the general population.

These are related to issues related to for example haematological toxicity of ¹⁷⁷Lu-lilotomab satetraxetan, and to finding the optimal way of having higher activity levels of ¹⁷⁷Lu-lilotomab satetraxetan in order to obtain a higher probability of obtaining partial or complete responses in the patients.

These challenges have now surprisingly been overcome.

### SUMMARY OF THE INVENTION

An aspect of the present invention relates to ¹⁷⁷Lu-lilotomab satetraxetan for use in the treatment of Non-Hodgkin lymphoma, wherein ¹⁷⁷Lu-lilotomab satetraxetan is administered according to an administration pattern comprising predosing of 20-250 mg/m² lilotomab, followed by 10-50 MBq/kg ¹⁷⁷Lu-lilotomab satetraxetan to a person in need thereof.

Another aspect of the present invention relates to lilotomab for use in the treatment of Non-Hodgkin lymphoma wherein lilotomab is administered according to an administration pattern comprising predosing of 20-250 mg/m² lilotomab, followed by 10-50 MBq/kg ¹⁷⁷Lu-lilotomab satetraxetan to a person in need thereof.

A further aspect of the present invention relates to a combination of lilotomab and ¹⁷⁷Lu-lilotomab satetraxetan for use in the treatment of Non-Hodgkin lymphoma wherein lilotomab and ¹⁷⁷Lu-lilotomab satetraxetan is administered according to an administration pattern comprising predosing of 20-250 mg/m² lilotomab, followed by 10-50 MBq/kg ¹⁷⁷Lu-lilotomab satetraxetan to a person in need thereof.

Yet another aspect of the present invention relates to lilotomab for use in the reduction of haematological toxicity due to the administration of ¹⁷⁷Lu-lilotomab satetraxetan, wherein lilotomab is administered before ¹⁷⁷Lu-lilotomab satetraxetan in a dose of 20-250 mg/m².

Another aspect of the present invention relates to a method of treating Non-Hodgkin lymphoma comprising administration of ¹⁷⁷Lu-lilotomab satetraxetan in an administration pattern comprising predosing of 20-250 mg/m² lilotomab, followed by 10-50 MBq/kg ¹⁷⁷Lu-lilotomab satetraxetan to a person in need thereof.

An embodiment of the present invention relates to ¹⁷⁷Lu-lilotomab satetraxetan administered at a concentration selected from the group consisting of 10, 12.5, 15, 17.5, 20, 25, 30, 35, 40, 45 and 50 MBq/kg.

Another embodiment of the present invention relates to lilotomab administered at a concentration selected from the group consisting of 20, 40, 50, 60, 75, 100, 125, 150, 200, 250 mg/m².

A further embodiment of the present invention relates to the predosing of lilotomab done less than 24 hours, such as within 4 hours, before administration of ¹⁷⁷Lu-lilotomab satetraxetan.

Another embodiment of the present invention relates to the uses and methods of the present invention further comprising a pretreatment step before predosing wherein the pretreatment step comprises pretreatment with one, two, three or more infusions of 375 mg/m² rituximab.

Yet another embodiment of the present invention relates to 375 mg/m² rituximab infused at 28 and 21 days before administration of ¹⁷⁷Lu-lilotomab satetraxetan.

A further embodiment of the present invention relates to 375 mg/m² rituximab infused at 14 days before administration of ¹⁷⁷Lu-lilotomab satetraxetan.

Another embodiment of the present invention relates to 375 mg/m² rituximab infused at 14 days and within 4 hours before administration of ¹⁷⁷Lu-lilotomab satetraxetan.

Another embodiment of the present invention relates to the lymphoma being a subtype selected from the group consisting of follicular grade I-IIIA, marginal zone, small lymphocytic, lymphoplasmacytic, and mantle cell.

### BRIEF DESCRIPTION OF THE FIGURES

FIG. 1 shows platelet counts of arm 3 (rituximab predosing) and arm 4 (100 mg/m² lilotomab predosing). The patients in arm 3 suffers grade 3-4 hematological toxicity while there is no toxicity in arm 4.

FIG. 2 shows neutrophil counts of arm 1 (40 mg lilotomab predosing), arm 3 (rituximab predosing) and arm 4 (100 mg/m² lilotomab predosing). The patients in arm 3 suffers grade 3-4 toxicity, while there is no toxicity of arm 4 and arm 1 is in between.

FIG. 3 shows PK profiles that show a large separation between the treatment arms. Arm 1=40 mg lilotomab predosing, Arm 2=no predosing, Arm 3=rituximab predosing and Arm 4=100 mg/m² lilotomab predosing.

FIG. 4 shows an example of an administration pattern.

FIG. 5 shows platelet counts of patients in arm 1 (40 mg predosing), arm 2 (no predosing), arm 3 (rituximab predosing) and arm 4 (100 mg/m² lilotomab predosing). There was grade 3-4 toxicity of arm 2 and 3, less toxicity of arm 1 and no toxicity of arm 4.

FIG. 6 shows neutrophil counts of patients in arm 1 (40 mg predosing), arm 2 (no predosing), arm 3 (rituximab predosing) and arm 4 (100 mg/m² lilotomab predosing). There was grade 3-4 toxicity of arm 2 and 3, less toxicity of arm 1 and no toxicity of arm 4.

FIG. 7 shows PK profiles that show a large separation between the treatment arms. Arm 1=40 mg lilotomab predosing, Arm 2=no predosing, Arm 3=rituximab predosing and Arm 4=100 mg/m² lilotomab predosing.

FIG. 8 shows examples of an administration patterns tested.

FIG. 9 shows that the mean values for platelets and neutrophils at nadir for 23 arm 1 patients were lower than the mean values for 3 arm 4 patients.

FIG. 10 shows dose limiting toxicity and number of grade 3 and 4 adverse events were lower for arm 4 than for arm 1 and highest for arm 2.

FIG. 11 shows the response rates for each to the tested administration patterns.

FIG. 12 shows four different combinations of pre-dosing and pre-treatment that have been investigated. Two arms included cold lilotomab pre-dosing (arm 1 and 4; 40 mg fixed and 100 mg/m² Body Surface Area dosage, respectively) and two did not (arm 2 and 3). Pre-dosing with lilotomab has a mitigating effect on red marrow absorbed dose for ¹⁷⁷Lu-lilotomab satetraxetan patients, and increased amounts was found correlated with a higher tumour dose.

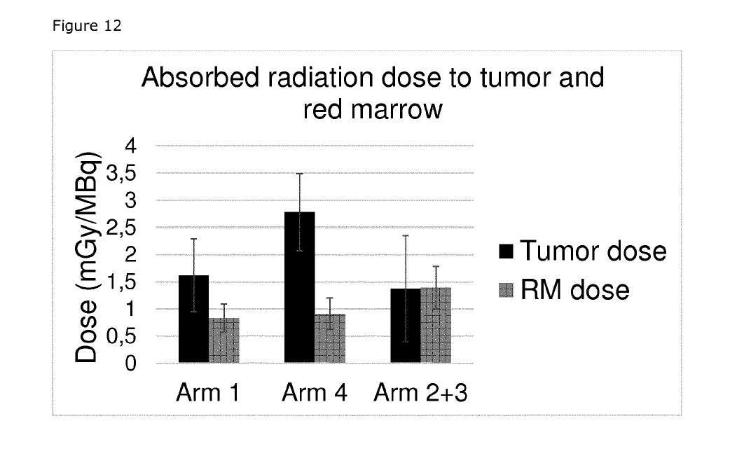

FIG. 13 shows mean platelet count in Arms 1 and 4 for 15 and 20 MBq/kg ¹⁷⁷Lu-lilotomab satetraxetan.

FIG. 14 shows mean neutrophil count in Arms 1 and 4 for 15 and 20 MBq/kg ¹⁷⁷Lu-lilotomab satetraxetan.

### DETAILED DESCRIPTION OF THE INVENTION

The present invention relates to the treatment of Non-Hodgkin lymphoma using ¹⁷⁷Lu-lilotomab satetraxetan with lilotomab and with or without rituximab, where the inventors surprisingly have found that a specific treatment pattern have advantageous effects.

An aspect of the present invention relates to ¹⁷⁷Lu-lilotomab satetraxetan for use in the treatment of Non-Hodgkin lymphoma or other CD37 positive blood cancers, wherein ¹⁷⁷Lu-lilotomab satetraxetan is administered according to a clinically relevant administration pattern comprising 10-20 MBq/kg ¹⁷⁷Lu-lilotomab satetraxetan, to a person in need thereof.

The clinically relevant administration pattern can be seen as an administration pattern that has clinical relevance and effect on human individuals suffering from Non-Hodgkin lymphoma.

A further aspect of the present invention relates to ¹⁷⁷Lu-lilotomab satetraxetan for use in the treatment of Non-Hodgkin lymphoma, wherein ¹⁷⁷Lu-lilotomab satetraxetan is administered according to an administration pattern comprising: a) predosing of 20-100 mg/m² lilotomab, followed by b) 10-20 MBq/kg ¹⁷⁷Lu-lilotomab satetraxetan to a person in need thereof.

In the present context can the term treatment be seen as the partial of full treatment of cancer, including any amelioration and for example stabilization of progressing disease.

Thus, the term treatment may be seen as an improvement of any of the criteria tested in the examples of the present disclosure. One criteria is overall response rate (ORR). Another is complete response (CR). A further is partial response (PR). Another is stable disease (SD).

The lilotomab predosing effect is likely caused by blocking of the binding on remaining B-cells in the lymphoid organs. This can be more effective after rituximab treatment.

Pre-medication consisting of an antipyretic and antihistamine medication can be administered before infusion of lilotomab.

Thus, an aspect of the present invention relates to ¹⁷⁷Lu-lilotomab satetraxetan and lilotomab for use the treatment of Non-Hodgkin lymphoma, wherein ¹⁷⁷Lu-lilotomab satetraxetan is administered according to a specific administration pattern.

Method of Treatment and for Use in Treatment

An aspect of the present invention relates to ¹⁷⁷Lu-lilotomab satetraxetan for use in the treatment of Non-Hodgkin lymphoma, wherein ¹⁷⁷Lu-lilotomab satetraxetan is administered according to an administration pattern comprising predosing of 20-250 mg/m² lilotomab, followed by 10-50 MBq/kg ¹⁷⁷Lu-lilotomab satetraxetan to a person in need thereof.

The predosing of 20-250 mg/m² lilotomab may be substituted with 40-500 mg/patient in all aspects and embodiments of the invention.

A further aspect of the present invention relates to lilotomab for use in the treatment of Non-Hodgkin lymphoma wherein lilotomab is administered according to an administration pattern comprising predosing of 20-250 mg/m² lilotomab, followed by 10-50 MBq/kg ¹⁷⁷Lu-lilotomab satetraxetan to a person in need thereof.

A further aspect of the present invention relates to a combination of lilotomab and ¹⁷⁷Lu-lilotomab satetraxetan for use in the treatment of Non-Hodgkin lymphoma wherein lilotomab and ¹⁷⁷Lu-lilotomab satetraxetan is administered according to an administration pattern comprising predosing of 20-250 mg/m² lilotomab, followed by 10-50 MBq/kg ¹⁷⁷Lu-lilotomab satetraxetan to a person in need thereof.

Yet another aspect of the present invention relates to lilotomab for use in the reduction of haematological toxicity due to the administration of ¹⁷⁷Lu-lilotomab satetraxetan, wherein lilotomab is administered before ¹⁷⁷Lu-lilotomab satetraxetan in a dose of 20-250 mg/m².

Another aspect of the present invention relates to a method of treating Non-Hodgkin lymphoma comprising administration of ¹⁷⁷Lu-lilotomab satetraxetan in an administration pattern comprising predosing of 20-250 mg/m² lilotomab, followed by 10-50 MBq/kg ¹⁷⁷Lu-lilotomab satetraxetan to a person in need thereof.

The therapy or treatment of the present invention can be administered either as a monotherapy or in combination with other therapies, preferentially standard treatments.

Such other therapies may be one or more selected from the group consisting of pretreatment, surgery, chemotherapy (including doxorubicin, vinblastin and gemcitabine), immunotherapy, antibody therapy, photodynamic therapy, proteasome inhibitor (including bortezomib), histone deacetylase inhibitors (including vorinostat and suberoylanilide hydroxamic acid), vitamin D3 and vitamin D3 analogs, cell cycle checkpoint inhibitors (including UCN-01 and 2-(4-(4-Chlorophenoxy)phenyl)-1H-benzimidazole-5-carboxamide), hypoxic cell radiosensitizers (including metronidazole and misonidazole), apoptosis inducers (including withaferin A and venetoclax), radiosensitizers, radioimmunotherapy or a combination of two or more of these.

In one embodiment of the present invention has the patient being treated according to the present invention already been undergoing treatment for cancer.

In one embodiment of the present invention is this treatment of therapy one or more of those mentioned above. In a preferred embodiment is the therapy rituximab, and in this case can the patient be a patient relapsing after rituximab treatment.

Thus, an embodiment of the present invention relates to ¹⁷⁷Lu-lilotomab satetraxetan for use in the treatment of Non-Hodgkin lymphoma according to the present invention, wherein the patient is relapsing after treatment with rituximab.

Lilotomab and ¹⁷⁷Lu-Lilotomab Satetraxetan

The monoclonal antibody (mAb or moAb) lilotomab was previously known as tetulomab or HH1 while ¹⁷⁷Lu-lilotomab satetraxetan was previously known as ¹⁷⁷Lu-labeled HH1 antibody, or named ¹⁷⁷Lu-tetulomab or by the tradename Betalutin.

¹⁷⁷Lu-lilotomab satetraxetan is a radioimmunoconjugate (RIC) also known as antibody radionuclide conjugate (ARC) that is capable of binding to or targeting an antigen of interest. In the present case is this antigen CD37.

Satetraxetan is a derivative of DOTA, p-SCN-benzyl-DOTA.

Administration Route

By administered is meant intravenous infusion or intravenous injection. More specifically, the radioimmunoconjugate and antibody of the present invention can be administered directly in a vein by a peripheral cannula connected to a drip chamber that prevents air embolism and allows an estimate of flow rate into the patient.

In one embodiment the radioimmunoconjugate and/or antibody can be administered in a repeated fashion.

In another embodiment the radioimmunoconjugate followed by monoclonal antibody (or immunoconjugate) can both be administered in a repeated fashion.

An embodiment of the present invention relates to the use of the radioimmunoconjugate and/or antibody of the present invention administered in combination with or in addition to other therapy.

In an embodiment of the present invention the other therapies are selected from pretreatment, chemotherapy, monoclonal antibody therapy, surgery, radiotherapy, and/or photodynamic therapy.

In another embodiment of the present invention the other therapies are bone marrow transplantation or stem cell transplantation and/or therapy.

Administration Dosages

In the present invention is ¹⁷⁷Lu-lilotomab satetraxetan used in the treatment of Non-Hodgkin's lymphoma. An embodiment of the present invention relates to ¹⁷⁷Lu-lilotomab satetraxetan administered at a concentration selected from the group consisting of 10, 12.5, 15, 17.5, 20, 25, 30, 35, 40, 45, 50 MBq/kg.

An embodiment of the present invention relates to ¹⁷⁷Lu-lilotomab satetraxetan administered at a concentration of 10 MBq/kg.

Another embodiment of the present invention relates to ¹⁷⁷Lu-lilotomab satetraxetan administered at a concentration of 12.5 MBq/kg.

A further embodiment of the present invention relates to ¹⁷⁷Lu-lilotomab satetraxetan administered at a concentration of 15 MBq/kg.

Yet another embodiment of the present invention relates to ¹⁷⁷Lu-lilotomab satetraxetan administered at a concentration of 17.5 MBq/kg.

Another embodiment of the present invention relates to ¹⁷⁷Lu-lilotomab satetraxetan administered at a concentration of 20 MBq/kg.

Another embodiment of the present invention relates to ¹⁷⁷Lu-lilotomab satetraxetan administered at a concentration of 25 MBq/kg.

Yet another embodiment of the present invention relates to ¹⁷⁷Lu-lilotomab satetraxetan administered at a concentration of 17.5-20 MBq/kg.

Yet another embodiment of the present invention relates to ¹⁷⁷Lu-lilotomab satetraxetan administered at a concentration of 20-25 MBq/kg.

Yet another embodiment of the present invention relates to ¹⁷⁷Lu-lilotomab satetraxetan administered at a concentration of 25-30 MBq/kg.

Yet another embodiment of the present invention relates to ¹⁷⁷Lu-lilotomab satetraxetan administered at a concentration of 30-35 MBq/kg.

Yet another embodiment of the present invention relates to ¹⁷⁷Lu-lilotomab satetraxetan administered at a concentration of 35-40 MBq/kg.

Yet another embodiment of the present invention relates to ¹⁷⁷Lu-lilotomab satetraxetan administered at a concentration of 40-45 MBq/kg.

Yet another embodiment of the present invention relates to ¹⁷⁷Lu-lilotomab satetraxetan administered at a concentration of 45-50 MBq/kg.

Lilotomab is used for predosing before administration of ¹⁷⁷Lu-lilotomab satetraxetan.

An embodiment of the present invention relates to lilotomab administered at a concentration of 40 mg/patient.

Another embodiment of the present invention relates to lilotomab administered at a concentration of 2-50 mg/patient.

Another embodiment of the present invention relates to lilotomab administered at a concentration of 40 mg/patient.

Another embodiment of the present invention relates to lilotomab administered at a concentration of 100 mg/patient.

Another embodiment of the present invention relates to lilotomab administered at a concentration of 120 mg/patient.

Another embodiment of the present invention relates to lilotomab administered at a concentration of 150 mg/patient.

Another embodiment of the present invention relates to lilotomab administered at a concentration of 200 mg/patient.

Another embodiment of the present invention relates to lilotomab administered at a concentration of 20 mg/m².

Another embodiment of the present invention relates to lilotomab administered at a concentration of 40 mg/m².

Another embodiment of the present invention relates to lilotomab administered at a concentration of 60 mg/m².

Another embodiment of the present invention relates to predosing of lilotomab administered at a concentration of 20 mg/m² followed by ¹⁷⁷Lu-lilotomab satetraxetan administered at a concentration of 15 MBq/kg.

Another embodiment of the present invention relates to predosing of lilotomab administered at a concentration of 20 mg/m² followed by ¹⁷⁷Lu-lilotomab satetraxetan administered at a concentration of 17.5 MBq/kg.

Another embodiment of the present invention relates to predosing of lilotomab administered at a concentration of 20 mg/m² followed by ¹⁷⁷Lu-lilotomab satetraxetan administered at a concentration of 20 MBq/kg.

Another embodiment of the present invention relates to predosing of lilotomab administered at a concentration of 40 mg/m² followed by ¹⁷⁷Lu-lilotomab satetraxetan administered at a concentration of 15 MBq/kg.

Another embodiment of the present invention relates to predosing of lilotomab administered at a concentration of 40 mg/m² followed by ¹⁷⁷Lu-lilotomab satetraxetan administered at a concentration of 17.5 MBq/kg.

Another embodiment of the present invention relates to predosing of lilotomab administered at a concentration of 100 mg/m² followed by ¹⁷⁷Lu-lilotomab satetraxetan administered at a concentration of 15 MBq/kg.

Another embodiment of the present invention relates to predosing of lilotomab administered at a concentration of 100 mg/m² followed by ¹⁷⁷Lu-lilotomab satetraxetan administered at a concentration of 17.5 MBq/kg.

Another embodiment of the present invention relates to predosing of lilotomab administered at a concentration of 40 mg/m² followed by ¹⁷⁷Lu-lilotomab satetraxetan administered at a concentration of 20 MBq/kg.

Another embodiment of the present invention relates to predosing of lilotomab administered at a concentration of 100 mg/m² followed by ¹⁷⁷Lu-lilotomab satetraxetan administered at a concentration of 20 MBq/kg.

Another embodiment of the present invention relates to predosing of lilotomab administered at a concentration of 40 mg/patient followed by ¹⁷⁷Lu-lilotomab satetraxetan administered at a concentration of 15 MBq/kg.

Another embodiment of the present invention relates to predosing of lilotomab administered at a concentration of 40 mg/patient followed by ¹⁷⁷Lu-lilotomab satetraxetan administered at a concentration of 17.5 MBq/kg.

Another embodiment of the present invention relates to predosing of lilotomab administered at a concentration of 40 mg/patient followed by ¹⁷⁷Lu-lilotomab satetraxetan administered at a concentration of 17.5 MBq/kg.

Another embodiment of the present invention relates to predosing of lilotomab administered at a concentration of 100 mg/patient followed by ¹⁷⁷Lu-lilotomab satetraxetan administered at a concentration of 15 MBq/kg.

Another embodiment of the present invention relates to predosing of lilotomab administered at a concentration of 40 mg/patient followed by ¹⁷⁷Lu-lilotomab satetraxetan administered at a concentration of 20 MBq/kg.

Another embodiment of the present invention relates to predosing of lilotomab administered at a concentration of 100 mg/patient followed by ¹⁷⁷Lu-lilotomab satetraxetan administered at a concentration of 20 MBq/kg.

Another embodiment of the present invention relates to predosing of lilotomab administered at a concentration of 50 mg/patient followed by ¹⁷⁷Lu-lilotomab satetraxetan administered at a concentration of 20 MBq/kg.

Another embodiment of the present invention relates to predosing of lilotomab administered at a concentration of 60 mg/patient followed by ¹⁷⁷Lu-lilotomab satetraxetan administered at a concentration of 20 MBq/kg.

A further embodiment of the present invention relates to lilotomab administered at a concentration of 50 mg/m². This may in an embodiment of the invention be equal to 100 mg/patient.

A further embodiment of the present invention relates to lilotomab administered at a concentration of 60 mg/m². This may in an embodiment of the invention be equal to 120 mg/patient.

Another embodiment of the present invention relates to lilotomab administered at a concentration of 75 mg/m². This may in an embodiment of the invention be equal to 150 mg/patient.

Yet another embodiment of the present invention relates to lilotomab administered at a concentration of 100 mg/m². This may in an embodiment of the invention be equal to 200 mg/patient.

Another embodiment of the present invention relates to lilotomab administered at a concentration of 125 mg/m². This may in an embodiment of the invention be equal to 250 mg/patient.

A further embodiment of the present invention relates to lilotomab administered at a concentration of 150 mg/m². This may in an embodiment of the invention be equal to 300 mg/patient. Another embodiment of the present invention relates to lilotomab administered at a concentration of 175 mg/m². This may in an embodiment of the invention be equal to 350 mg/patient.

A further embodiment of the present invention relates to lilotomab administered at a concentration of 200 mg/m². This may in an embodiment of the invention be equal to 400 mg/patient. Another embodiment of the present invention relates to lilotomab administered at a concentration of 225 mg/m². This may in an embodiment of the invention be equal to 450 mg/patient.

Another embodiment of the present invention relates to lilotomab administered at a concentration of 40 mg/patient.

Another embodiment of the present invention relates to lilotomab administered at a concentration of 50 mg/patient.

Another embodiment of the present invention relates to lilotomab administered at a concentration of 60 mg/patient.

Yet another embodiment of the present invention relates to lilotomab administered at a concentration of 250 mg/m². This may in an embodiment of the invention be equal to 500 mg/patient. Another embodiment of the present invention relates to lilotomab administered at a concentration of 20-250 mg/m². This may in an embodiment of the invention be equal to 10-125 mg/patient. A further embodiment of the present invention relates to lilotomab administered at a concentration of 20-100 mg/m². This may in an embodiment of the invention be equal to 40-200 mg/patient.

Another embodiment of the present invention relates to lilotomab administered at a concentration of 20-150 mg/m². This may in an embodiment of the invention be equal to 40-300 mg/patient.

A further embodiment of the present invention relates to lilotomab administered at a concentration of 100-200 mg/m². This may in an embodiment of the invention be equal to 200-400 mg/patient.

Another embodiment of the present invention relates to ¹⁷⁷Lu-lilotomab satetraxetan administered at a concentration of 15-20 MBq/kg and lilotomab administered at a concentration of 20-100 mg/m².

Yet another embodiment of the present invention relates to ¹⁷⁷Lu-lilotomab satetraxetan administered at a concentration of 15-20 MBq/kg and lilotomab administered at a concentration of 40-100 mg/m². This may in an embodiment of the invention be equal to 80-200 mg/patient.

Another embodiment of the present invention relates to ¹⁷⁷W-lilotomab satetraxetan administered at a concentration of 17.5-20 MBq/kg and lilotomab administered at a concentration of 40-100 mg/m².

A further embodiment of the present invention relates to ¹⁷⁷W-lilotomab satetraxetan administered at a concentration of 15 MBq/kg and lilotomab administered at a concentration of 100 mg/m².

Another embodiment of the present invention relates to ¹⁷⁷Lu-lilotomab satetraxetan administered at a concentration of 17.5 MBq/kg and lilotomab administered at a concentration of 100 mg/m².

Yet another embodiment of the present invention relates to ¹⁷⁷Lu-lilotomab satetraxetan administered at a concentration of 20 MBq/kg and lilotomab administered at a concentration of 100 mg/m².

Yet another embodiment of the present invention relates to ¹⁷⁷Lu-lilotomab satetraxetan administered at a concentration of 25 MBq/kg and lilotomab administered at a concentration of 100 mg/m².

Yet another embodiment of the present invention relates to ¹⁷⁷Lu-lilotomab satetraxetan administered at a concentration of 30 MBq/kg and lilotomab administered at a concentration of 100 mg/m².

Yet another embodiment of the present invention relates to ¹⁷⁷Lu-lilotomab satetraxetan administered at a concentration of 35 MBq/kg and lilotomab administered at a concentration of 100 mg/m².

Yet another embodiment of the present invention relates to ¹⁷⁷Lu-lilotomab satetraxetan administered at a concentration of 40 MBq/kg and lilotomab administered at a concentration of 100 mg/m².

Yet another embodiment of the present invention relates to ¹⁷⁷Lu-lilotomab satetraxetan administered at a concentration of 45 MBq/kg and lilotomab administered at a concentration of 100 mg/m².

Yet another embodiment of the present invention relates to ¹⁷⁷Lu-lilotomab satetraxetan administered at a concentration of 50 MBq/kg and lilotomab administered at a concentration of 100 mg/m².

Yet another embodiment of the present invention relates to ¹⁷⁷Lu-lilotomab satetraxetan administered at a concentration of 15 MBq/kg and lilotomab administered at a concentration of 60 mg/m².

Yet another embodiment of the present invention relates to ¹⁷⁷Lu-lilotomab satetraxetan administered at a concentration of 20 MBq/kg and lilotomab administered at a concentration of 60 mg/m².

Yet another embodiment of the present invention relates to ¹⁷⁷Lu-lilotomab satetraxetan administered at a concentration of 25 MBq/kg and lilotomab administered at a concentration of 60 mg/m².

Yet another embodiment of the present invention relates to ¹⁷⁷Lu-lilotomab satetraxetan administered at a concentration of 30 MBq/kg and lilotomab administered at a concentration of 60 mg/m².

A further embodiment of the present invention relates to ¹⁷⁷Lu-lilotomab satetraxetan administered at a concentration of 15 MBq/kg and lilotomab administered at a concentration of 40-100 mg/m².

Another embodiment of the present invention relates to ¹⁷⁷Lu-lilotomab satetraxetan administered at a concentration of 17.5 MBq/kg and lilotomab administered at a concentration of 40-100 mg/m².

Yet another embodiment of the present invention relates to ¹⁷⁷Lu-lilotomab satetraxetan administered at a concentration of 20 MBq/kg and lilotomab administered at a concentration of 40-100 mg/m².

A further embodiment of the present invention relates to ¹⁷⁷Lu-lilotomab satetraxetan administered at a concentration of 25 MBq/kg and lilotomab administered at a concentration of 40-100 mg/m².

Another embodiment of the present invention relates to ¹⁷⁷Lu-lilotomab satetraxetan administered at a concentration of 30 MBq/kg and lilotomab administered at a concentration of 40-100 mg/m².

Yet another embodiment of the present invention relates to ¹⁷⁷Lu-lilotomab satetraxetan administered at a concentration of 35 MBq/kg and lilotomab administered at a concentration of 40-100 mg/m².

A further embodiment of the present invention relates to ¹⁷⁷Lu-lilotomab satetraxetan administered at a concentration of 40 MBq/kg and lilotomab administered at a concentration of 40-100 mg/m².

Another embodiment of the present invention relates to ¹⁷⁷Lu-lilotomab satetraxetan administered at a concentration of 45 MBq/kg and lilotomab administered at a concentration of 40-100 mg/m².

Yet another embodiment of the present invention relates to ¹⁷⁷Lu-lilotomab satetraxetan administered at a concentration of 50 MBq/kg and lilotomab administered at a concentration of 40-100 mg/m².

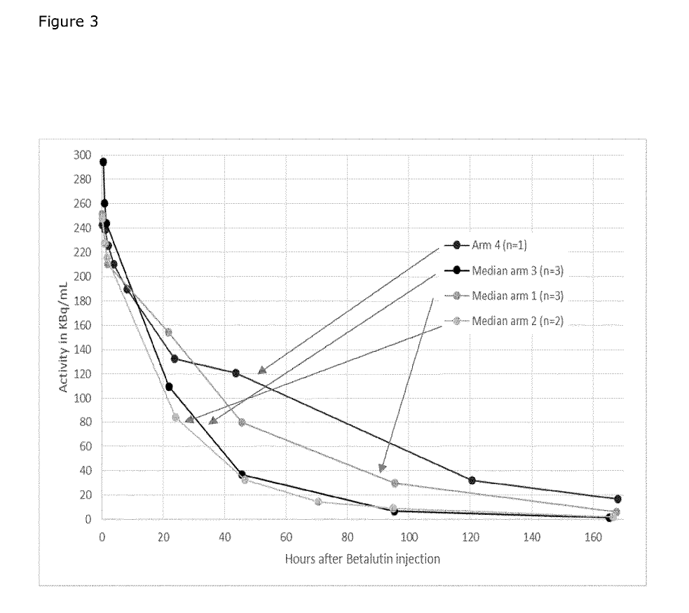

The PK profiles (e.g. FIG. 3) show activity in kBq/ml in the hours after ¹⁷⁷Lu-lilotomab satetraxetan administration. A high concentration means that a high amount of ¹⁷⁷Lu-lilotomab satetraxetan is present in the blood.

Thus, in one embodiment of the present invention is the activity (in kBq/ml) after 72 hours more than 80 kBq/ml, such as more than 70 kBq/ml, such as more than 60 kBq/ml.

In another embodiment of the present invention is the activity (in kBq/ml) after 48 hours more than 110 kBq/ml, such as more than 100 kBq/ml, such as more than 90 kBq/ml, such as more than 80 kBq/ml.

Haematological Toxicity

The administration of immunosuppressive agents may be associated with the occurrence of hematologic toxicity, such as anemia, due to bone marrow suppression or hemolysis, leukopenia, neutropenia and thrombocytopenia.

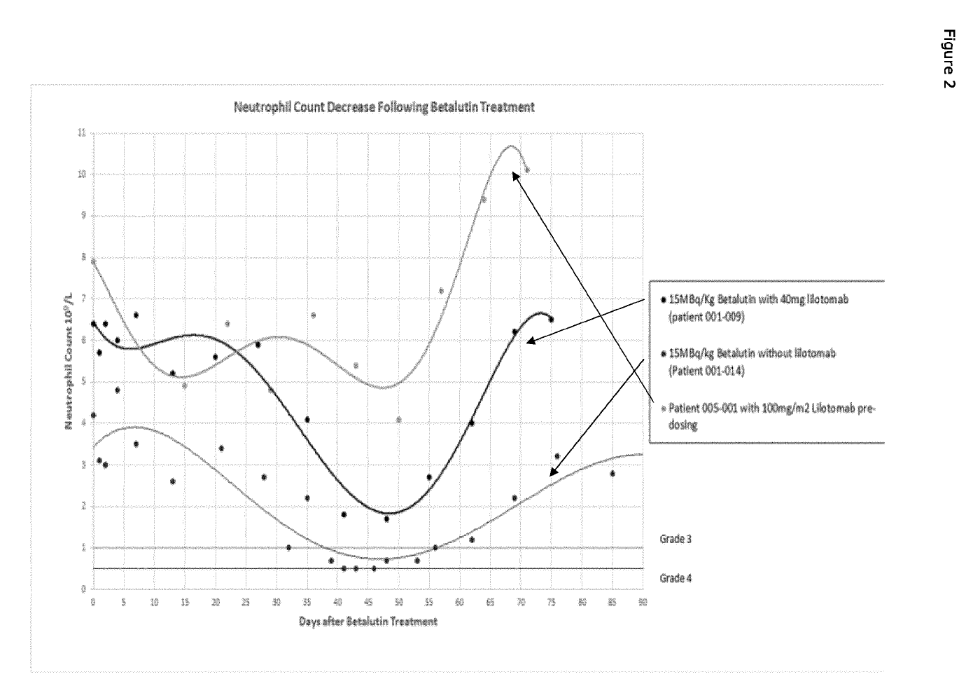

Neutropenia is graded; grade 1 is Neutrophils <LLN to 1500/mm³, grade 2 is Neutrophils <1500/mm³ to 1000/mm³, grade 3 is Neutrophils <1000/mm³ to 500/mm³, and grade 4 is Neutrophils <500/mm³ (see also FIG. 2)

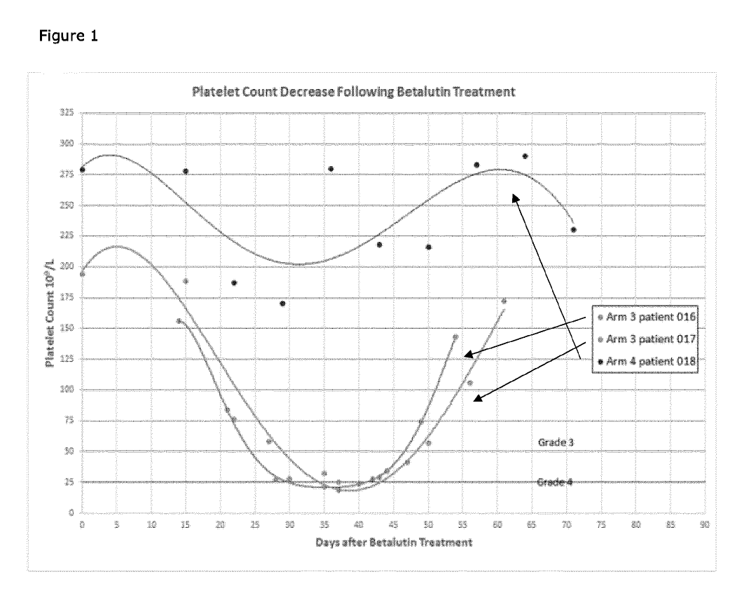

Thrombocytopenia is graded; grade 1 is Platelets <LLN to 75,000/mm³, grade 2 is <75,000/mm³ to 50,000/mm³, grade 3 is <50,000/mm³ to 25,000/mm³, and grade 4 is <25,000/mm³ (see also FIG. 1).

Preferably is no neutropenia grade 4 observed after the treatment, and even more preferably is no grade 3 or 4 observed 45 days after the treatment.

Preferably is no thrombocytopenia grade 4 observed after the treatment, and even more preferably is no grade 3 or 4 observed 45 days after the treatment.

In one embodiment is no grade 3 or 4 neutropenia and thrombocytopenia or no grade 3 neutropenia and thrombocytopenia observed 45 days after the treatment.

Neutropenia and thrombocytopenia in patients can for example be seen in example 1 and FIGS. 1 and 2.

An aspect of the present invention relates to the use of lilotomab to reduce hematologic toxicity such as neutropenia and/or thrombocytopenia in patients suffering from non-Hodgkin's lymphoma. These patients may previously be treated with rituximab. The patients may also subsequently be treated with ¹⁷⁷Lu-lilotomab satetraxetan as disclosed herein.

Administration Timing

As noted above the radioimmunoconjugates and/or antibody can be used in combination with other types of therapy.

Thus, in a further embodiment of the present invention is the use for a combinational therapy where the radioimmunoconjugate followed by simultaneous or post-treatment with antibody therapy, immunoconjugate therapy or a combination thereof, as described elsewhere herein.

Such therapy or treatment may be a monoclonal antibody selected from rituximab and lilotomab (HH1) depending on the antigen in focus.

The therapy can be repeated in cyclic pattern where administration of the radioimmunoconjugates and the monoclonal antibodies are repeated once, twice or several times.

A further embodiment of the present invention relates to the predosing of lilotomab done less than 24 hours, such as within 4 hours before administration of ¹⁷⁷Lu-lilotomab satetraxetan.

Another embodiment of the present invention relates to the predosing of lilotomab done less than 12 hours before administration of ¹⁷⁷Lu-lilotomab satetraxetan.

A further embodiment of the present invention relates to the predosing of lilotomab done less than 8 hours before administration of ¹⁷⁷Lu-lilotomab satetraxetan.

Yet another embodiment of the present invention relates to the predosing of lilotomab done less than 4 hours before administration of ¹⁷⁷Lu-lilotomab satetraxetan.

A further embodiment of the present invention relates to the predosing of lilotomab done less than 2 hours before administration of ¹⁷⁷Lu-lilotomab satetraxetan.

Rituximab Administration

Rituximab is a monoclonal antibody against the protein CD20, which is primarily found on the surface of immune system B cells.

Another embodiment of the present invention relates to the uses and methods of the present invention further comprising a pretreatment step before predosing wherein the pretreatment step comprises pretreatment with one, two, three or more injections or infusions of 375 mg/m² rituximab.

Another embodiment of the present invention relates to the uses and methods of the present invention further comprising a pretreatment step before predosing wherein the pretreatment step comprises pretreatment with one injection or infusion of 375 mg/m² rituximab.

Another embodiment of the present invention relates to the uses and methods of the present invention further comprising a pretreatment step before predosing wherein the pretreatment step comprises pretreatment with two injections or infusions of 375 mg/m² rituximab.

Another embodiment of the present invention relates to the uses and methods of the present invention further comprising a pretreatment step before predosing wherein the pretreatment step comprises pretreatment with three or more injections or infusions of 375 mg/m² rituximab.

Another embodiment of the present invention relates to the uses and methods of the present invention further comprising a pretreatment step before predosing wherein the pretreatment step comprises pretreatment with one, two, three or more injections or infusions of 100-750 mg/m² rituximab.

Another embodiment of the present invention relates to the uses and methods of the present invention further comprising a pretreatment step before predosing wherein the pretreatment step comprises pretreatment with one, two, three or more injections or infusions of 200-750 mg/m² rituximab.

Another embodiment of the present invention relates to the uses and methods of the present invention further comprising a pretreatment step before predosing wherein the pretreatment step comprises pretreatment with one, two, three or more injections or infusions of 300-700 mg/m² rituximab.

Another embodiment of the present invention relates to the uses and methods of the present invention further comprising a pretreatment step before predosing wherein the pretreatment step comprises pretreatment with 375 mg/m² rituximab. This treatment can be repeated, once, twice or several times.

Rituximab can be injected or infused. The pretreatment can be done 28-7 days before administration of ¹⁷⁷Lu-lilotomab satetraxetan.

Another embodiment of the present invention relates to rituximab infused or injected once or twice 28-14 days before administration of ¹⁷⁷Lu-lilotomab satetraxetan. An additional infusion or injection of rituximab can be done less than 4 hours before administration of ¹⁷⁷Lu-lilotomab satetraxetan.

Another embodiment of the present invention relates to rituximab infused or injected once or twice 10-18 days before administration of ¹⁷⁷Lu-lilotomab satetraxetan.

Another embodiment of the present invention relates to rituximab infused or injected once, twice, or three times at day 28, 21 or 14 before administration of ¹⁷⁷Lu-lilotomab satetraxetan.

Yet another embodiment of the present invention relates to 375 mg/m² rituximab infused or injected at 28 and 21 days before administration of ¹⁷⁷Lu-lilotomab satetraxetan.

A further embodiment of the present invention relates to 375 mg/m² rituximab infused or injected at 14 days before administration of ¹⁷⁷Lu-lilotomab satetraxetan.

Another embodiment of the present invention relates to 375 mg/m² rituximab infused or injected at 14 days and again less than 4 hours before administration of ¹⁷⁷Lu-lilotomab satetraxetan.

Specific Administration Patterns

An aspect of the present invention relates to ¹⁷⁷Lu-lilotomab satetraxetan for use in the treatment of Non-Hodgkin lymphoma, wherein ¹⁷⁷Lu-lilotomab satetraxetan is administered according in an administration pattern comprising predosing of 40 mg/patient of lilotomab, followed by 15 MBq/kg ¹⁷⁷Lu-lilotomab satetraxetan to a person in need thereof.

An aspect of the present invention relates to ¹⁷⁷Lu-lilotomab satetraxetan for use in the treatment of Non-Hodgkin lymphoma, wherein ¹⁷⁷Lu-lilotomab satetraxetan is administered according in an administration pattern comprising predosing of 100 mg/m² lilotomab, followed by 15 MBq/kg ¹⁷⁷Lu-lilotomab satetraxetan to a person in need thereof. Another aspect of the present invention relates to ¹⁷⁷Lu-lilotomab satetraxetan for use in the treatment of Non-Hodgkin lymphoma, wherein ¹⁷⁷Lu-lilotomab satetraxetan is administered according in an administration pattern comprising predosing of 40 mg/m² lilotomab, followed by 17.5 MBq/kg ¹⁷⁷W-lilotomab satetraxetan to a person in need thereof.

A further aspect of the present invention relates to ¹⁷⁷Lu-lilotomab satetraxetan for use in the treatment of Non-Hodgkin lymphoma, wherein ¹⁷⁷W-lilotomab satetraxetan is administered according in an administration pattern comprising predosing of 60 mg/m² lilotomab, followed by 20 MBq/kg ¹⁷⁷Lu-lilotomab satetraxetan to a person in need thereof.

A further aspect of the present invention relates to ¹⁷⁷Lu-lilotomab satetraxetan for use in the treatment of Non-Hodgkin lymphoma, wherein ¹⁷⁷W-lilotomab satetraxetan is administered according to an administration pattern comprising predosing of 100 mg/m² lilotomab, followed by 25 MBq/kg ¹⁷⁷W-lilotomab satetraxetan to a person in need thereof.

A further aspect of the present invention relates to ¹⁷⁷Lu-lilotomab satetraxetan for use in the treatment of Non-Hodgkin lymphoma, wherein ¹⁷⁷Lu-lilotomab satetraxetan is administered according in an administration pattern comprising predosing of 100 mg/m² lilotomab, followed by 30 MBq/kg ¹⁷⁷W-lilotomab satetraxetan to a person in need thereof.

A further aspect of the present invention relates to ¹⁷⁷Lu-lilotomab satetraxetan for use in the treatment of Non-Hodgkin lymphoma, wherein ¹⁷⁷W-lilotomab satetraxetan is administered according in an administration pattern comprising predosing of 100 mg/m² lilotomab, followed by 35 MBq/kg ¹⁷⁷Lu-lilotomab satetraxetan to a person in need thereof.

A further aspect of the present invention relates to ¹⁷⁷Lu-lilotomab satetraxetan for use in the treatment of Non-Hodgkin lymphoma, wherein ¹⁷⁷Lu-lilotomab satetraxetan is administered according in an administration pattern comprising predosing of 100 mg/m² lilotomab, followed by 45 MBq/kg ¹⁷⁷Lu-lilotomab satetraxetan to a person in need thereof.

A further aspect of the present invention relates to ¹⁷⁷Lu-lilotomab satetraxetan for use in the treatment of Non-Hodgkin lymphoma, wherein ¹⁷⁷Lu-lilotomab satetraxetan is administered according in an administration pattern comprising predosing of 100 mg/m² lilotomab, followed by 50 MBq/kg ¹⁷⁷Lu-lilotomab satetraxetan to a person in need thereof.

An aspect of the present invention relates to ¹⁷⁷Lu-lilotomab satetraxetan for use the treatment of Non-Hodgkin lymphoma, wherein ¹⁷⁷Lu-lilotomab satetraxetan is administered according in an administration pattern comprising predosing of 100 mg/m² lilotomab, followed by 15 MBq/kg ¹⁷⁷Lu-lilotomab satetraxetan to a person in need thereof.

Another aspect of the present invention relates to ¹⁷⁷Lu-lilotomab satetraxetan for use the treatment of Non-Hodgkin lymphoma, wherein ¹⁷⁷Lu-lilotomab satetraxetan is administered according in an administration pattern comprising predosing of 100 mg/m² lilotomab, followed by 17.5 MBq/kg ¹⁷⁷Lu-lilotomab satetraxetan to a person in need thereof.

An aspect of the present invention relates to ¹⁷⁷Lu-lilotomab satetraxetan for use the treatment of Non-Hodgkin lymphoma, wherein ¹⁷⁷Lu-lilotomab satetraxetan is administered according in an administration pattern comprising predosing of 100 mg/m² lilotomab, followed by 20 MBq/kg ¹⁷⁷Lu-lilotomab satetraxetan to a person in need thereof.

An aspect of the present invention relates to ¹⁷⁷Lu-lilotomab satetraxetan for use the treatment of Non-Hodgkin lymphoma, wherein ¹⁷⁷Lu-lilotomab satetraxetan is administered according in an administration pattern comprising predosing of 100 mg/m² lilotomab, followed by 25 MBq/kg ¹⁷⁷Lu-lilotomab satetraxetan to a person in need thereof.

An aspect of the present invention relates to ¹⁷⁷Lu-lilotomab satetraxetan for use the treatment of Non-Hodgkin lymphoma, wherein ¹⁷⁷Lu-lilotomab satetraxetan is administered according in an administration pattern comprising predosing of 100 mg/m² lilotomab, followed by 30 MBq/kg ¹⁷⁷Lu-lilotomab satetraxetan to a person in need thereof.

An aspect of the present invention relates to ¹⁷⁷Lu-lilotomab satetraxetan for use in the treatment of Non-Hodgkin lymphoma, wherein ¹⁷⁷Lu-lilotomab satetraxetan is administered according in an administration pattern comprising pretreatment using 375 mg/m² rituximab 14 days prior to administration of ¹⁷⁷Lu-lilotomab satetraxetan, predosing of 100 mg/m² lilotomab, followed by 15 MBq/kg ¹⁷⁷Lu-lilotomab satetraxetan to a person in need thereof.

An aspect of the present invention relates to ¹⁷⁷Lu-lilotomab satetraxetan for use the treatment of Non-Hodgkin lymphoma, wherein ¹⁷⁷Lu-lilotomab satetraxetan is administered according in an administration pattern comprising pretreatment using 375 mg/m² rituximab 14 days prior to administration of ¹⁷⁷Lu-lilotomab satetraxetan, predosing of 100 mg/m² lilotomab, followed by 17.5 MBq/kg ¹⁷⁷Lu-lilotomab satetraxetan to a person in need thereof.

An aspect of the present invention relates to ¹⁷⁷Lu-lilotomab satetraxetan for use the treatment of Non-Hodgkin lymphoma, wherein ¹⁷⁷Lu-lilotomab satetraxetan is administered according in an administration pattern comprising pretreatment using 375 mg/m² rituximab 14 days prior to administration of ¹⁷⁷Lu-lilotomab satetraxetan, predosing of 100 mg/m² lilotomab, followed by 20 MBq/kg ¹⁷⁷Lu-lilotomab satetraxetan to a person in need thereof.

An aspect of the present invention relates to ¹⁷⁷Lu-lilotomab satetraxetan for use the treatment of Non-Hodgkin lymphoma, wherein ¹⁷⁷Lu-lilotomab satetraxetan is administered according in an administration pattern comprising pretreatment using 375 mg/m² rituximab 14 days prior to administration of ¹⁷⁷Lu-lilotomab satetraxetan, predosing of 100 mg/m² lilotomab, followed by 25 MBq/kg ¹⁷⁷Lu-lilotomab satetraxetan to a person in need thereof.

An aspect of the present invention relates to ¹⁷⁷Lu-lilotomab satetraxetan for use the treatment of Non-Hodgkin lymphoma, wherein ¹⁷⁷Lu-lilotomab satetraxetan is administered according in an administration pattern comprising pretreatment using 375 mg/m² rituximab 14 days prior to administration of ¹⁷⁷Lu-lilotomab satetraxetan, predosing of 100 mg/m² lilotomab, followed by 30 MBq/kg ¹⁷⁷Lu-lilotomab satetraxetan to a person in need thereof.

Another aspect of the present invention relates to ¹⁷⁷Lu-lilotomab satetraxetan for use the treatment of Non-Hodgkin lymphoma, wherein ¹⁷⁷Lu-lilotomab satetraxetan is administered according in an administration pattern comprising pretreatment using 375 mg/m² rituximab 14 days before administration of ¹⁷⁷Lu-lilotomab satetraxetan and again less than 4 hours before administration of ¹⁷⁷Lu-lilotomab satetraxetan, predosing of 100 mg/m² lilotomab less than 4 hours before administration of ¹⁷⁷Lu-lilotomab satetraxetan, followed by 15 MBq/kg ¹⁷⁷Lu-lilotomab satetraxetan to a person in need thereof.

Another aspect of the present invention relates to ¹⁷⁷Lu-lilotomab satetraxetan for use the treatment of Non-Hodgkin lymphoma, wherein ¹⁷⁷Lu-lilotomab satetraxetan is administered according in an administration pattern comprising pretreatment using 375 mg/m² rituximab 14 days before administration of ¹⁷⁷Lu-lilotomab satetraxetan and again less than 4 hours before administration of ¹⁷⁷Lu-lilotomab satetraxetan, predosing of 100 mg/m² lilotomab less than 4 hours before administration of ¹⁷⁷Lu-lilotomab satetraxetan, followed by 17.5 MBq/kg ¹⁷⁷Lu-lilotomab satetraxetan to a person in need thereof.

A further aspect of the present invention relates to ¹⁷⁷Lu-lilotomab satetraxetan for use the treatment of Non-Hodgkin lymphoma, wherein ¹⁷⁷Lu-lilotomab satetraxetan is administered according in an administration pattern comprising pretreatment using 375 mg/m² rituximab 14 days before administration of ¹⁷⁷Lu-lilotomab satetraxetan and again less than 4 hours before administration of ¹⁷⁷Lu-lilotomab satetraxetan, predosing of 100 mg/m² lilotomab less than 4 hours before administration of 177Lu-lilotomab satetraxetan, followed by 20 MBq/kg ¹⁷⁷Lu-lilotomab satetraxetan to a person in need thereof.

A further aspect of the present invention relates to ¹⁷⁷Lu-lilotomab satetraxetan for use the treatment of Non-Hodgkin lymphoma, wherein ¹⁷⁷W-lilotomab satetraxetan is administered according in an administration pattern comprising pretreatment using 375 mg/m² rituximab 14 days before administration of ¹⁷⁷Lu-lilotomab satetraxetan and again less than 4 hours before administration of ¹⁷⁷Lu-lilotomab satetraxetan, predosing of 100 mg/m² lilotomab less than 4 hours before administration of ¹⁷⁷Lu-lilotomab satetraxetan, followed by 25 MBq/kg ¹⁷⁷Lu-lilotomab satetraxetan to a person in need thereof.

A further aspect of the present invention relates to ¹⁷⁷Lu-lilotomab satetraxetan for use the treatment of Non-Hodgkin lymphoma, wherein ¹⁷⁷Lu-lilotomab satetraxetan is administered according in an administration pattern comprising pretreatment using 375 mg/m² rituximab 14 days before administration of ¹⁷⁷Lu-lilotomab satetraxetan and again less than 4 hours before administration of ¹⁷⁷Lu-lilotomab satetraxetan, predosing of 100 mg/m² lilotomab less than 4 hours before administration of ¹⁷⁷Lu-lilotomab satetraxetan, followed by 30 MBq/kg ¹⁷⁷Lu-lilotomab satetraxetan to a person in need thereof.

Another aspect of the present invention relates to lilotomab for use in the treatment of Non-Hodgkin lymphoma wherein lilotomab is administered according to an administration pattern comprising pretreatment using 375 mg/m² rituximab 14 days before administration of ¹⁷⁷Lu-lilotomab satetraxetan and again less than 4 hours before administration of ¹⁷⁷Lu-lilotomab satetraxetan, predosing of 100 mg/m² lilotomab less than 4 hours before administration of ¹⁷⁷Lu-lilotomab satetraxetan, followed by 15 MBq/kg ¹⁷⁷Lu-lilotomab satetraxetan to a person in need thereof.

A further aspect of the present invention relates to a combination of lilotomab and ¹⁷⁷Lu-lilotomab satetraxetan for use in the treatment of Non-Hodgkin lymphoma wherein lilotomab and ¹⁷⁷Lu-lilotomab satetraxetan is administered according to an administration pattern comprising pretreatment using 375 mg/m² rituximab 14 days before administration of ¹⁷⁷Lu-lilotomab satetraxetan and again less than 4 hours before administration of ¹⁷⁷Lu-lilotomab satetraxetan, predosing of 100 mg/m² lilotomab less than 4 hours before administration of ¹⁷⁷Lu-lilotomab satetraxetan, followed by 15 MBq/kg ¹⁷⁷Lu-lilotomab satetraxetan to a person in need thereof.

A further aspect of the present invention relates to a combination of lilotomab and ¹⁷⁷Lu-lilotomab satetraxetan for use in the treatment of Non-Hodgkin lymphoma wherein lilotomab and ¹⁷⁷Lu-lilotomab satetraxetan is administered according to an administration pattern comprising pretreatment using 375 mg/m² rituximab 14 days before administration of ¹⁷⁷Lu-lilotomab satetraxetan and again less than 4 hours before administration of ¹⁷⁷Lu-lilotomab satetraxetan, predosing of 100 mg/m² lilotomab less than 4 hours before administration of ¹⁷⁷Lu-lilotomab satetraxetan, followed by 17.5 MBq/kg ¹⁷⁷Lu-lilotomab satetraxetan to a person in need thereof.

A further aspect of the present invention relates to a combination of lilotomab and ¹⁷⁷Lu-lilotomab satetraxetan for use in the treatment of Non-Hodgkin lymphoma wherein lilotomab and ¹⁷⁷Lu-lilotomab satetraxetan is administered according to an administration pattern comprising pretreatment using 375 mg/m² rituximab 14 days before administration of ¹⁷⁷Lu-lilotomab satetraxetan and again less than 4 hours before administration of ¹⁷⁷Lu-lilotomab satetraxetan, predosing of 100 mg/m² lilotomab less than 4 hours before administration of ¹⁷⁷Lu-lilotomab satetraxetan, followed by 20 MBq/kg ¹⁷⁷Lu-lilotomab satetraxetan to a person in need thereof.

A further aspect of the present invention relates to a combination of lilotomab and ¹⁷⁷Lu-lilotomab satetraxetan for use in the treatment of Non-Hodgkin lymphoma wherein lilotomab and ¹⁷⁷Lu-lilotomab satetraxetan is administered according to an administration pattern comprising pretreatment using 375 mg/m² rituximab 14 days before administration of ¹⁷⁷Lu-lilotomab satetraxetan and again less than 4 hours before administration of ¹⁷⁷Lu-lilotomab satetraxetan, predosing of 100 mg/m² lilotomab less than 4 hours before administration of ¹⁷⁷Lu-lilotomab satetraxetan, followed by 25 MBq/kg ¹⁷⁷Lu-lilotomab satetraxetan to a person in need thereof.

A further aspect of the present invention relates to a combination of lilotomab and ¹⁷⁷Lu-lilotomab satetraxetan for use in the treatment of Non-Hodgkin lymphoma wherein lilotomab and ¹⁷⁷Lu-lilotomab satetraxetan is administered according to an administration pattern comprising pretreatment using 375 mg/m² rituximab 14 days before administration of ¹⁷⁷W-lilotomab satetraxetan and again less than 4 hours before administration of ¹⁷⁷Lu-lilotomab satetraxetan, predosing of 100 mg/m² lilotomab less than 4 hours before administration of ¹⁷⁷Lu-lilotomab satetraxetan, followed by 30 MBq/kg ¹⁷⁷Lu-lilotomab satetraxetan to a person in need thereof.

Yet another aspect of the present invention relates to lilotomab for use in the reduction of haematological toxicity due to the administration of ¹⁷⁷Lu-lilotomab satetraxetan, wherein lilotomab is administered before ¹⁷⁷Lu-lilotomab satetraxetan in a dose of 100 mg/m².

Another aspect of the present invention relates to a method of treating Non-Hodgkin lymphoma comprising administration of pretreatment using 375 mg/m² rituximab 14 days before administration of ¹⁷⁷Lu-lilotomab satetraxetan and again less than 4 hours before administration of ¹⁷⁷Lu-lilotomab satetraxetan, predosing of 100 mg/m² lilotomab less than 4 hours before administration of ¹⁷⁷Lu-lilotomab satetraxetan, followed by 15 MBq/kg ¹⁷⁷Lu-lilotomab satetraxetan to a person in need thereof.

Another aspect of the present invention relates to a method of treating Non-Hodgkin lymphoma comprising administration of pretreatment using 375 mg/m² rituximab 14 days before administration of ¹⁷⁷Lu-lilotomab satetraxetan and again less than 4 hours before administration of ¹⁷⁷Lu-lilotomab satetraxetan, predosing of 100 mg/m² lilotomab less than 4 hours before administration of ¹⁷⁷Lu-lilotomab satetraxetan, followed by 17.5 MBq/kg ¹⁷⁷Lu-lilotomab satetraxetan to a person in need thereof.

Another aspect of the present invention relates to a method of treating Non-Hodgkin lymphoma comprising administration of pretreatment using 375 mg/m² rituximab 14 days before administration of ¹⁷⁷Lu-lilotomab satetraxetan and again less than 4 hours before administration of ¹⁷⁷Lu-lilotomab satetraxetan, predosing of 100 mg/m² lilotomab less than 4 hours before administration of ¹⁷⁷Lu-lilotomab satetraxetan, followed by 20 MBq/kg ¹⁷⁷Lu-lilotomab satetraxetan to a person in need thereof.

Another aspect of the present invention relates to a method of treating Non-Hodgkin lymphoma comprising administration of pretreatment using 375 mg/m² rituximab 14 days before administration of ¹⁷⁷Lu-lilotomab satetraxetan and again less than 4 hours before administration of ¹⁷⁷Lu-lilotomab satetraxetan, predosing of 100 mg/m² lilotomab less than 4 hours before administration of ¹⁷⁷Lu-lilotomab satetraxetan, followed by 25 MBq/kg ¹⁷⁷Lu-lilotomab satetraxetan to a person in need thereof.

Another aspect of the present invention relates to a method of treating Non-Hodgkin lymphoma comprising administration of pretreatment using 375 mg/m² rituximab 14 days before administration of ¹⁷⁷Lu-lilotomab satetraxetan and again less than 4 hours before administration of ¹⁷⁷Lu-lilotomab satetraxetan, predosing of 100 mg/m² lilotomab less than 4 hours before administration of ¹⁷⁷Lu-lilotomab satetraxetan, followed by 30 MBq/kg ¹⁷⁷Lu-lilotomab satetraxetan to a person in need thereof.

The therapy or treatment of the present invention can be administered either as a monotherapy or in combination with other therapies, preferentially standard treatments.

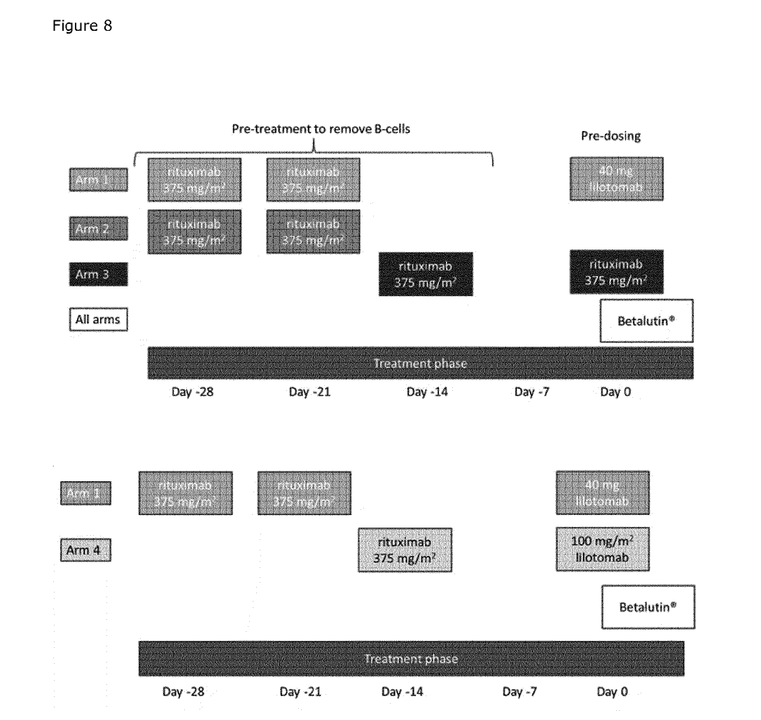

An aspect of the present invention relates to the treatment patterns shown in Arm 1 of FIG. 8.

An aspect of the present invention relates to the treatment patterns shown in Arm 2 of FIG. 8.

An aspect of the present invention relates to the treatment patterns shown in Arm 3 of FIG. 8.

An aspect of the present invention relates to the treatment patterns shown in Arm 4 of FIG. 8.

Pre-medication consisting of an antipyretic and antihistamine medication can be administered before infusion of rituximab. The types of pre-medication are in accordance with each hospital's routine, including any use of corticosteroids.

Pharmaceutical Compositions

Antibodies and radioimmunoconjugates are usually applied in the treatment of diseases formulated in pharmaceutical compositions.

Such compositions are optimized for parameters such as physiological tolerance and shelf-life.

Thus, in one embodiment of the present invention is the radioimmunoconjugates and/or antibodies of the present invention formulated as a pharmaceutical composition.

An embodiment of the present invention relates to a pharmaceutical composition as described above, further comprising one or more additional therapeutic agents.

In another embodiment of the present invention are said one or more additional therapeutic agents selected from agents that induce apoptosis.

Usually is an important element of a pharmaceutical composition a buffer solution, which to a substantial degree maintain the chemical integrity of the radioimmunoconjugate and/or antibody and is being physiologically acceptable for infusion into patients.

In one embodiment of the present invention the pharmaceutical composition comprises one or more pharmaceutically acceptable carriers and/or adjuvants.

Acceptable pharmaceutical carriers include but are not limited to non-toxic buffers, fillers, isotonic solutions, etc. More specifically, the pharmaceutical carrier can be but are not limited to normal saline (0.9%), half-normal saline, Ringer's lactate, 5%) Dextrose, 3.3% Dextrose/0.3% Saline. The physiologically acceptable carrier can contain a radiolytic stabilizer, e.g., ascorbic acid, which protect the integrity of the radiopharmaceutical during storage and shipment.

In one embodiment of the present invention are lilotomab and Betalutin formulated as indicated in Tables 1 and 2 in Example 1.

Preferably are sodium dihydrogen phosphate monohydrate, sodium chloride, recombinant human albumin, sodium ascorbate, diethylenetriamine pentaacetic acid (DTPA) and sodium hydroxide used as excipients in the formulation buffer.

Preferably is phosphate included in the formulation buffer to maintain the pH of the finished product during the shelf life.

Preferably is recombinant human albumin included in the formulation buffer as a stabilizer for the lilotomab satetraxetan conjugate. The albumin also acts as a radioprotectant. Recombinant human albumin structurally identical to human serum albumin derived from yeast is used. No human- or animal-derived raw material is involved in its manufacture. The excipient is well known and is used in pharmaceutical products for human use.

Preferably is sodium ascorbate included in the formulation to act as a radiolytic scavenger to ensure the stability of Betalutin over the shelf-life of the product.

Preferably is DTPA introduced as an excipient in the Betalutin formulation to chelate any free ¹⁷⁷Lu³⁺ ions and to reroute this impurity from accumulation in the bone to rapid renal clearance (Li et al 2001, Breeman et al 2003). Betalutin contains 9.3 μmol DTPA in 12 mL, while the maximum amount of no-carrier added (n.c.a) ¹⁷⁷Lu³⁺ (>3,000 GBq/mg) applied (6.9 GBq) corresponds to less than 15 nmol Lu ions. This gives a more than 1000-fold molar excess of DTPA over Lu³+ ions. Furthermore, when taking into account that the majority of the Lu3+ ions 95%) is chelated to lilotomab satetraxetan, the molar excess is almost 100,000-fold. DTPA is therefore expected to chelate all free ¹⁷⁷Lu³⁺ ions quantitatively and ¹⁷⁷Lu-DTPA is thus specified as radiochemical impurity in the specification.

Preferably is the formulation buffer an aqueous solution with pH 6.9 to 7.0 and thus no incompatibilities between the drug substance and the formulation buffer are expected.

One embodiment of the present invention comprises the pharmaceutical composition of the present invention and one or more additional antibodies or radioimmunoconjugates.

As aspect of the present invention relates to a pharmaceutical composition comprising (per mL): 0.75 mg Lutetium (¹⁷⁷Lu) lilotomab satetraxetan, 0.46 mg Ammonium acetate, and Trace amounts of HCl₃.

Another aspect of the present invention relates to a pharmaceutical composition comprising (per mL): 30.86 mg Sodium ascorbate, 0.31 mg DTPA, 0.17 mg NaOH, 60.82 mg Recombinant human albumin, 3.32 mg Sodium dihydrogen phosphate monohydrate, and 4.34 mg Sodium chloride with the pH is adjusted to 6.9-7.0.

A further aspect of the present invention relates to a pharmaceutical composition comprising;

14% of the pharmaceutical composition comprising (per mL): 0.75 mg Lutetium (¹⁷⁷Lu) lilotomab satetraxetan, 0.46 mg Ammonium acetate, and Trace amounts of HCl₃, and 86% of the pharmaceutical composition comprising (per mL): 30.86 mg Sodium ascorbate, 0.31 mg DTPA, 0.17 mg NaOH, 60.82 mg Recombinant human albumin, 3.32 mg Sodium dihydrogen phosphate monohydrate, and 4.34 mg Sodium chloride with the pH is adjusted to 6.9-7.0.

The present invention also relates to the pharmaceutical compositions of the present examples, as well as the dosage administration patterns presented herein. This includes the use of the pharmaceutical compositions of the present invention for use in the treatment of Non-Hodgkin lymphoma.

Cancer Types

The person in need of treatment with ¹⁷⁷Lu-lilotomab satetraxetan is suffering from a CD37 related disease, typically a B-cell lymphoma such as Non-Hodgkin lymphoma (NHL).

NHL is a group of blood cancers that includes all types of lymphoma except Hodgkin's lymphomas. Symptoms include enlarged lymph nodes, fever, night sweats, weight loss, and feeling tired. Other symptoms may include bone pain, chest pain, or itchiness. Some forms are slow growing while others are fast growing.

There are several types of NHL. Thus, another embodiment of the present invention relates to the lymphoma being a subtype selected from the group consisting of follicular grade I-IIIA, marginal zone, small lymphocytic, lymphoplasmacytic, Diffuse large B-cell lymphoma, and mantle cell.

In an embodiment of the present invention is the NHL cancer follicular grade I-IIIA.

In an embodiment of the present invention is the NHL cancer marginal zone.

In an embodiment of the present invention is the NHL cancer small lymphocytic.

In an embodiment of the present invention is the NHL cancer lymphoplasmacytic.

In an embodiment of the present invention is the NHL cancer mantle cell.

In an embodiment of the present invention is the NHL cancer AML.

In an embodiment of the present invention is the NHL cancer CLL.

In an embodiment of the present invention is the NHL cancer Diffuse Large B-cell lymphoma (DLBCL).

Some cell types of leukemia also express the CD37 antigen. Thus, another embodiment of the present invention relates to leukemia of the subtypes chronic lymphocytic leukemia and acute myelogen leukemia. More specifically the present invention relates to AML with 11Q23/MLL translocation. Thus, in one embodiment of the present invention is the NHL cancer AML with 11Q23/MLL translocation.

General

It should be understood that any feature and/or aspect discussed above in connections with the compounds and particles according to the invention apply by analogy to the methods and applications described herein.

The following figures and examples are provided below to illustrate the present invention. They are intended to be illustrative and are not to be construed as limiting in any way.

### EXAMPLES

### Example 1—Clinical Study on ¹⁷⁷Lu-Lilotomab Satetraxetan

Materials and Methods

Betalutin is an antibody-radionuclide-conjugate (ARC) composed of the radioisotope lutetium-177, the linker benzyl-DOTA and the murine anti-CD37 IgG1 antibody, lilotomab. The active moiety is the beta particle emitting nuclide ¹⁷⁷Lu. Lutetium-177 has physical half-life of 6.7 days. The antibody lilotomab recognises epitopes on the CD37 antigen, which is abundant on the cell surface of tumours of B-cell origin, including NHL. Betalutin is prepared as a solution for intravenous administration. 1 mg/ml lilotomab antibody will be used, between 7 to 20 mg lilotomab antibody per patient. The amount of lutetium (¹⁷⁷Lu)-lilotomab satetraxetan injected per patient will depend on dose level and patient's weight; however, the dose is capped for patients who weigh more than 130 kg (patients heavier than 130 kg will receive the dose for a 130 kg patient). Betalutin are supplied in vials containing a ready to use solution.

The investigational medicinal product will be referred to as Betalutin or lutetium (¹⁷⁷Lu)-lilotomab satetraxetan in the protocol.

Rituximab (MabThera) is used as pre-treatment. Rituximab, a chimeric anti-CD20 antibody will be used to clear the circulating normal peripheral B-lymphocytes in the blood and in the spleen before administrating CD37 targeting Betalutin. This may secure better access for Betalutin to less accessible compartments such as lymph nodes and larger tumour masses. Rituximab targets CD20 and will not block the binding of Betalutin CD37 on the B-lymphocytes or tumour cells. Betalutin contains a murine monoclonal antibody which has been shown from in vitro analysis to bind to the human Fc-γ receptor IIa. While rituximab binds to CD20 it also binds to the Fc-γ receptor IIa and if administered just prior to Betalutin may therefore inhibit the binding of Betalutin to this receptor and improve its biodistribution. Arm 3 has therefore been included in the study via a protocol amendment to test the ability of rituximab to improve the biodistribution of Betalutin. This improved biodistribution may reduce the incidence of myleosuppressive adverse events by decreasing the radioactivity in the bone marrow and spleen.

In Phase I arms 1, 2, and Phase II, two intravenous infusions of 375 mg/m² rituximab have been given, at 28 Days and 21 Days, before administration of Betalutin. In Phase I, arms 3, 4 and 5, one intravenous infusion of 375 mg/m2 rituximab have been given 14 days before, and in arms 3 and 5 an additional intravenous infusion of 375 mg/m² rituximab will be given within 4 hours before Betalutin administration on Day 0. Pre-medication consisting of an antipyretic and antihistamine medication should be administered before infusion of rituximab. The types of pre-medication are in accordance with each hospital's routine, including any use of corticosteroids.

Lilotomab is used as pre-dosing. The same antibody, lilotomab, as used in Betalutin, a murine anti-CD37 antibody, is used to block the binding on remaining B-cells, after rituximab treatment, in the lymphoid organs. One intravenous infusion of 40 mg lilotomab in arm 1, and 100 mg/m² lilotomab in arm 4 and arm 5, is performed within 4 hours before administration of Betalutin (up to a maximum of 2.7 m² for lilotomab in arm 4 and 5). Pre-medication consisting of an antipyretic and antihistamine medication should be administered before infusion of lilotomab.

Betalutin is administered in a dose of 15-20 Mbq/kg. Arm 4 is 15 Mbq/kg.

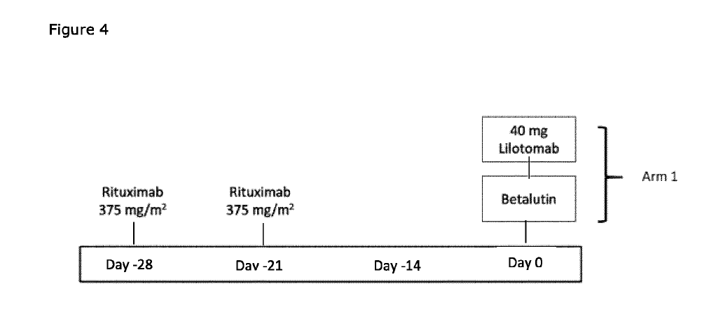

An example of administration pattern can be seen in FIG. 4 and the composition components are shown in tables 1 and 2.

TABLE 1
Composition of Betalutin Solution for Injection

| | | | |
|---|---|---|---|
| COMPOSITION | | | REFERENCE |
| FOR THE | QUANTITY | | TO |
| COMPONENTS | PER ML | FUNCTION | STANDARDS |

Drug substance

| | | | |
|---|---|---|---|
| Lutetium (¹⁷⁷Lu) | 0.75 mg | Drug substance | GMP |
| lilotomab | | | manufactured |
| satetraxetan | | | |
| Ammonium acetate | 0.46 mg | pH adjustment | Ph. Eur. |
| HCl³ | Trace | Solvent for ¹⁷⁷Lu | Ph. Eur./USP |

Formulation buffer

| | | | |
|---|---|---|---|
| Sodium ascorbate | 30.86 mg | Radiolytic | USP |
| | | scavenger | |
| DTPA | 0.31 mg | Chelation of | USP |
| | | free ¹⁷⁷Lu | |
| NaOH | 0.17 mg | pH adjustment | Ph. Eur./USP-NF |
| Recombinant human | 60.82 mg | Stabiliser/ | USP/NF |
| albumin | | radioprotectant | |
| Sodium dihydrogen | 3.32 mg | Buffer | USP/BP |
| phosphate | | | |
| monohydrate | | | |
| Sodium chloride | 4.34 mg | Osmolyte | USP |

TABLE 2
Composition of lilotomab Drug Product

| | | | | REFERENCE |
|---|---|---|---|---|
| | AMOUNT | | | TO |
| COMPONENT | PER ML | | FUNCTION | STANDARDS |
| Lilotomab drug substance, | | | | In house. |
| consisting of: | | | | |
| Lilotomab | 5 | mg | Active | |
| | | | ingredient | |
| Disodium hydrogen | 12.7 | mg | Buffer | |
| phosphate dodecahydrate | | | | |
| Sodium dihydrogen | 0.7 | mg | Buffer | |
| phosphate dihydrate | | | | |
| Sodium chloride | 0.5 | mg | Osmolyte | |
| Sucrose | 50 | mg | Stabilizer | |
| Polysorbate 20 | 0.2 | mg | Stabilizer | |
| WFI | Ad 1 | mL | Solvent | |

Results

These results are the results of a phase I/II clinical study on humans.

Platelet and neutrophil counts of arm 3 (rituximab predosing) and arm 4 (100 mg/m² lilotomab predosing) show grade 3-4 toxicity of arm 3 and no toxicity of arm 4 (FIGS. 1 and 2).

The PK profiles show a large separation between the treatment arms. Arm 1=40 mg lilotomab predosing, Arm 2=no predosing, Arm 3=rituximab predosing and Arm 4=100 mg/m² lilotomab predosing (FIG. 3).

### Example 2—Clinical Study on ¹⁷⁷Lu-Lilotomab Satetraxetan

Materials and Methods

Materials and methods are the same as in Example 1.

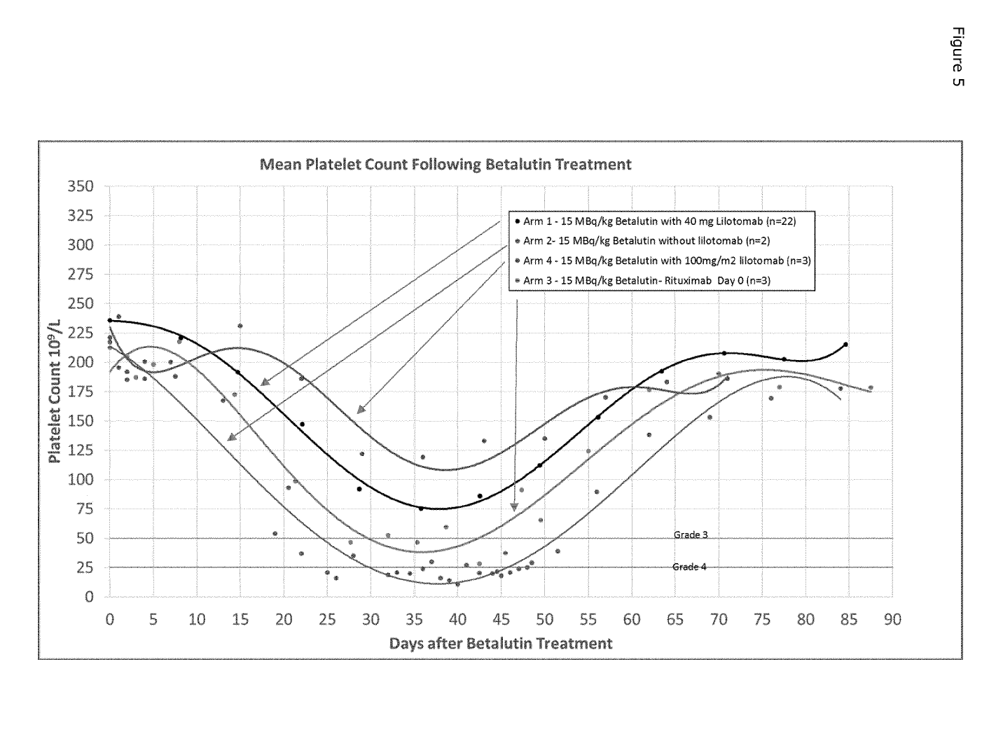

In Phase I arms 1, 2, and Phase II, two intravenous infusions of 375 mg/m2 rituximab have been given, at 28 Days and 21 Days, before administration of Betalutin (FIG. 5). In Phase I, arms 3 and 4, one intravenous infusion of 375 mg/m2 rituximab have been given 14 days before, and in arm 3 an additional intravenous infusion of 375 mg/m2 rituximab have been given within 4 hours before Betalutin administration on Day 0 (FIG. 5). Pre-medication consisting of an antipyretic and antihistamine medication should be administered before infusion of rituximab. The types of pre-medication are in accordance with each hospital's routine, including any use of corticosteroids.

Lilotomab is used as pre-dosing. The same antibody, lilotomab, as used in Betalutin, a murine anti-CD37 antibody, is used to block the binding on remaining B-cells, after rituximab treatment, in the lymphoid organs. One intravenous infusion of 40 mg lilotomab in arm 1, and 100 mg/m2 lilotomab in arm 4, is performed within 4 hours before administration of Betalutin (up to a maximum of 2.7 m2 for lilotomab in arm 4 and 5). Pre-medication consisting of an antipyretic and antihistamine medication should be administered before infusion of lilotomab.

Betalutin is administered in a dose of 15-20 MBq/kg.

Examples of administration patterns can be seen in FIG. 5 and the composition components are shown in tables 1 and 2 of example 1.

Results

These results are the results of a phase I/II clinical study on humans.

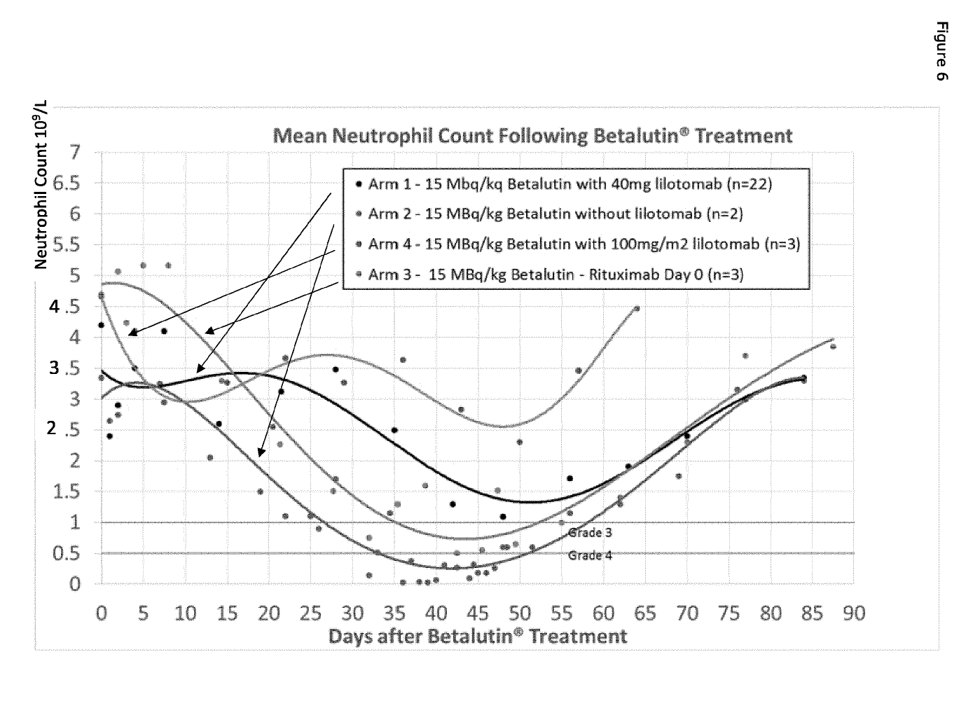

Platelet and neutrophil counts of patients in arm 1 (40 mg predosing), arm 2 (no predosing), arm 3 (rituximab predosing) and arm 4 (100 mg/m2 lilotomab predosing) show grade 3-4 toxicity of arm 2 and 3, less toxicity of arm 1 and no toxicity of arm 4 (FIGS. 5 and 6).

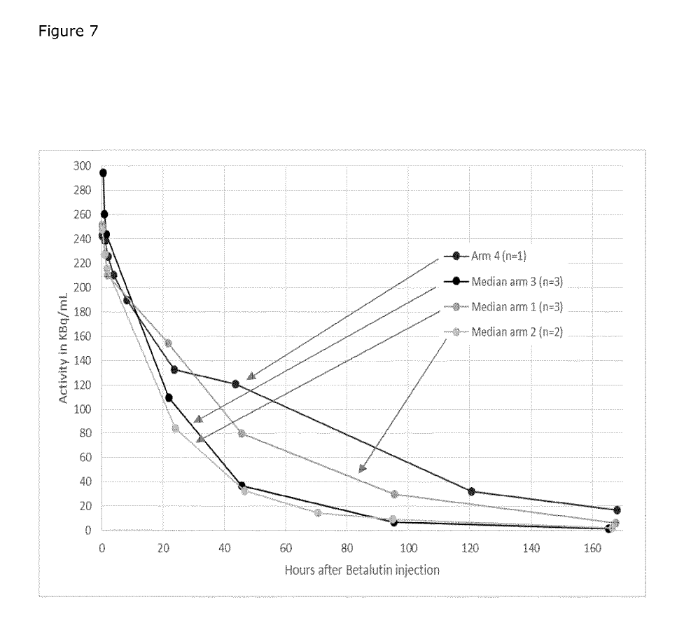

The PK profiles show a large separation between the treatment arms. Arm 1=40 mg lilotomab predosing, Arm 2=no predosing, Arm 3=rituximab predosing and Arm 4=100 mg/m2 lilotomab predosing (FIG. 7).

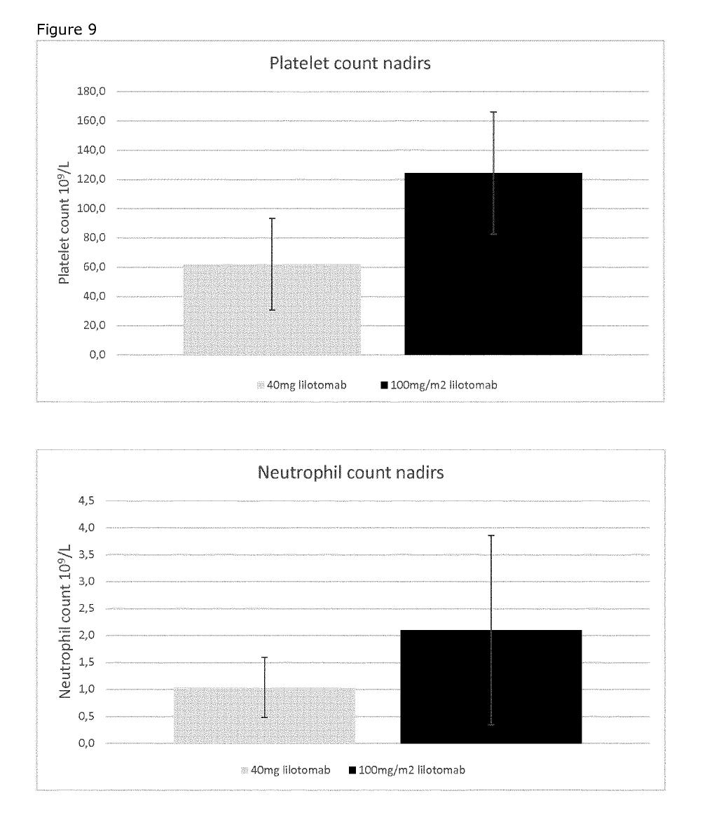

The mean values for platelets and neutrophils at nadir for 23 arm 1 patients were lower than the mean values for 3 arm 4 patients (FIG. 9).

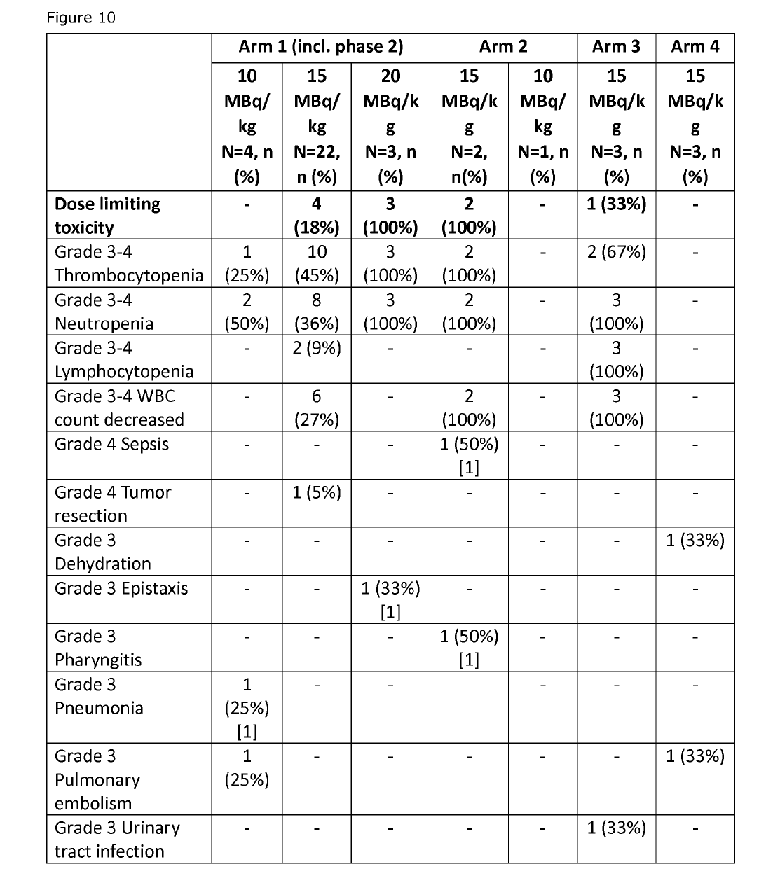

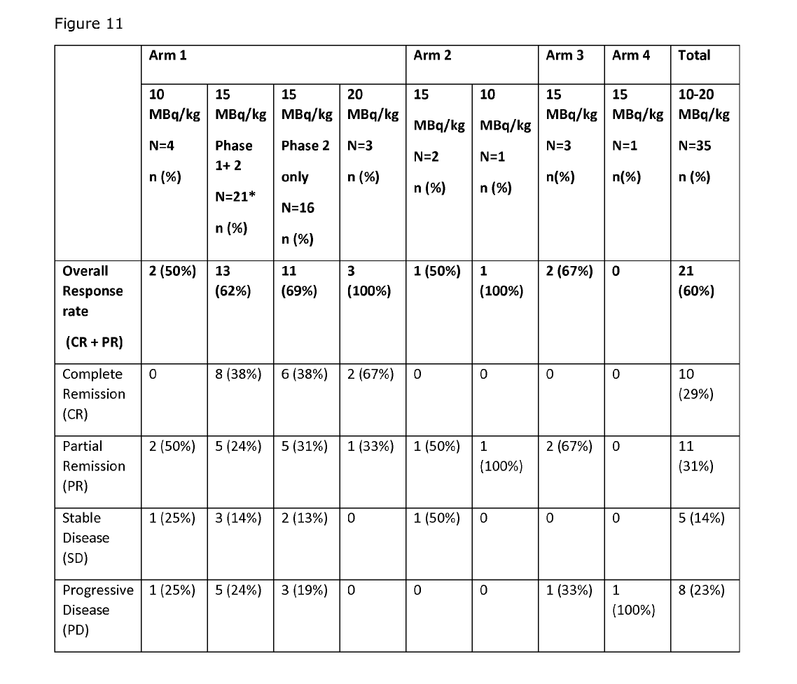

The dose limiting toxicity and number of grade 3 and 4 adverse events were lower for arm 4 than for arm 1 and highest for arm 2 (FIG. 10). The efficacy was equal for all arms (FIG. 11).

### Example 3—Pre-Dosing with Lilotomab Prior to Treatment with ¹⁷⁷Lu-Lilotomab Satetraxetan Significantly Increases the Ratio of Tumour to Red Marrow Absorbed Dose in Non-Hodgkin Lymphoma Patients

Aim:

Four different combinations of pre-dosing and pre-treatment have been investigated. All patients were pre-treated with different regimens of rituximab. Two arms included cold lilotomab pre-dosing (arm 1 and 4; 40 mg fixed and 100 mg/m² Body Surface Area dosage, respectively) and two did not (arm 2 and 3). Patients received either 10, 15 or 20 MBq ¹⁷⁷Lu-lilotomab satetraxetan per kg body weight. Previously, we have shown that absorbed red marrow (RM) doses were lower in arm1 vs arm2, and that haematological toxicity was more severe for patients receiving higher RM doses. The aim of this work was to compare the ratios of tumour to RM absorbed doses between arm 1, 4 and non-pre-dosed patients (arm 2+3).

Materials and Methods:

A total of 16 patients were included for RM dosimetry, of these were 14 included for tumour dosimetry. A total of 35 tumours were included, 1 to 5 from each patient (mode 3). RM and mean tumour absorbed doses per administered activity were determined from multiple SPECT/CT-images for each patient. Two-sided student-t-tests were used for all statistical analyses.

Results:

The mean RM absorbed doses were 0.83, 0.91 and 1.39 mGy/MBq for arm 1, 4 and non-pre-dosing respectively. There was a significantly higher RM dose for non-pre-dosing compared to arm 1 (p=0.04), and arm 4 (p=0.05). Mean tumour absorbed doses were 1.62, 2.78 and 1.37 mGy/MBq for arm 1, 4 and non-pre-dosing respectively. Tumour doses were higher in arm 4 patients compared to patients without pre-dosing (p=0.04). Tumour doses in arm 1 were not significantly higher compared to non-pre-dosing (p=0.71). The mean tumour to RM absorbed dose ratios were 2.16, 3.93 and 1.07 for arm 1, 4 and non-pre-dosing respectively. Ratios were significantly higher in both arm 1 and 4 compared to non-pre-dosing (p=0.05 and p=0.04). No statistically significant difference between arm 1 and 4 was found for any parameters (p>=0.12).

Conclusion:

Pre-dosing with lilotomab has a mitigating effect on red marrow absorbed dose for ¹⁷⁷Lu-lilotomab satetraxetan patients, and increased amounts was found correlated with a higher tumour dose. Both pre-dosage levels significantly increased the tumour to RM absorbed dose ratio.

### Example 4—Efficacy and Hematological Toxicity of ¹⁷⁷Lu-Lilotomab Satetraxetan in Non-Hodgkin Lymphoma Patients

Aim:

Four different combinations of pre-dosing and pre-treatment have been investigated. All patients were pre-treated with different regimens of 375 mg/m² rituximab. The patients were enrolled into four dose-escalation arms:
Arm 1: ¹⁷⁷Lu-lilotomab satetraxetan+pre-dosing with 40 mg lilotomab (cold anti-CD37 Ab)
Arm 2: ¹⁷⁷Lu-lilotomab satetraxetan without pre-dosing
Arm 3: ¹⁷⁷Lu-lilotomab satetraxetan+pre-dosing with 375 mg/m2 rituximab
Arm 4: ¹⁷⁷Lu-lilotomab satetraxetan+pre-dosing with 100 mg/m2 lilotomab

Patients received either 10, 15 or 20 MBq ¹⁷⁷Lu-lilotomab satetraxetan per kg body weight. The aim of this work was to determine the therapeutic efficacy and hematological toxicity of each study arm.

After finding the maximum tolerable dose (MTD), arm 1 was continued into a phase 2 part to evaluate efficacy in a larger data-set.

Patients and Methods:

The key eligibility criteria was: 1) Age ≥18 with histologically confirmed relapsed indolent B-cell NHL (follicular grade I-IIIA, mantle cell, SLL, marginal zone, lymphoplasmacytic subtypes). 2) <25% bone marrow involvement. 3) Life expectancy months. 4) Platelet count >150×10⁹/L. 5) ANC ≥1.5×10⁹/L. 6) No previous hematopoietic stem cell transplantation.

Dose-limiting toxicities (DLTs) were assessed during the first 12 weeks. Incidence and severity of adverse events (AEs) according to common terminology criteria for adverse events (CTCAE) v4.0. Response assessments were conducted at 3, 6 (FDG PET-CT), 9, 12, 18, 24 and 36 months (CT) per the International Working Group (IWG) criteria for NHL.

A total of 52 patients were included in the different arms and dose groups according to Table 1.

TABLE 1
Distribution of patients in different study arms and dose groups

| | 10 MBq/kg | 15 MBq/kg | 20 MBq/kg | Total |
|---|---|---|---|---|
| Arm 1 | 4 | 29 | 3 | 36 |
| Arm 2 | 1 | 2 | 0 | 3 |
| Arm 3 | 0 | 3 | 0 | 3 |
| Arm 4 | 0 | 3 | 7 | 10 |
| Total | 5 | 37 | 10 | 52 |

Safety

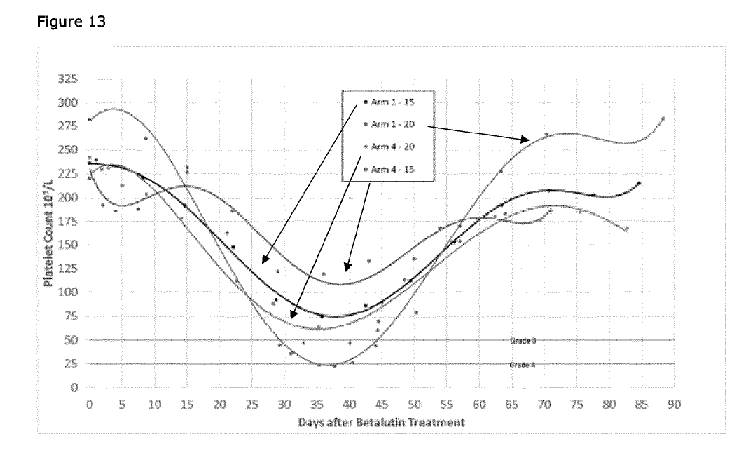

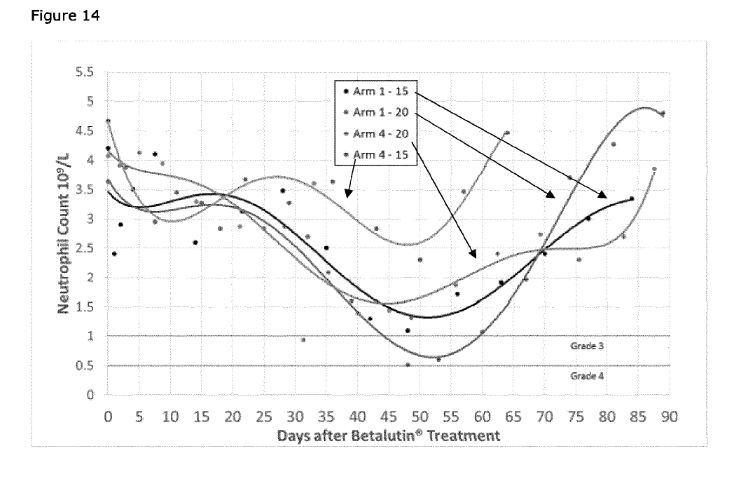

Overall, ¹⁷⁷Lu-lilotomab satetraxetan was well-tolerated. The most common grade 3/4 adverse events were reversible thrombocytopenia and neutropenia. No grade 4 neutropenia/thrombocytopenia was observed with higher lilotomab pre-dosing and 100 mg/m² gave a higher bone marrow protection than 40 mg (FIGS. 1, 2). Dose-limiting toxicities were prolonged but reversible neutropenia and thrombocytopenia (8 patients), and hematuria associated with thrombocytopenia (1 patient). The recommended dose for phase 2 expansion of ¹⁷⁷Lu-lilotomab satetraxetan in Arm 1 with a lilotomab pre-dose of 40 mg was 15 MBq/kg. Twenty-three patients were included in the current analysis (Table 1, arm 1, 15 MBq/kg). Arm 4, 20 MBq/kg was also selected for expansion into phase 2, but no patients have been included yet. SAEs occurred in 15 patients (25%). Treatment-emergent SAEs occurring in 2 or more patients were thrombocytopenia (n=2), atrial fibrillation (n=2), and lymphoma progression (n=2). Eighteen months after subsequent treatment with bendamustine (24 months after Betaluting), myelodysplastic syndrome (MDS) or acute myelogen leukemia (AML) was reported in 1 patient with prior alkylating agent exposure. There were no treatment-related deaths. Mean platelet count in Arms 1 and 4 for 15 and 20 MBq/kg 177Lu-lilotomab satetraxetan can be seen in FIG. 13 and mean neutrophil count in Arms 1 and 4 for 15 and 20 MBq/kg ¹⁷⁷Lu-lilotomab satetraxetan can be seen in FIG. 14.

Efficacy

Overall, objective responses were observed in 33 of 52 (63%) patients. 13 patients (25%) achieved a CR (Table 2). Significant activity was seen in patients with relapsed follicular lymphoma (FL) (ORR 70%; CR 24%). The ORR of arm 1, 2 and 3 were similar, while the ORR of arm 4 was lower. There was, however, too few patients in arm 2, 3 and 4 to draw any firm conclusions.

TABLE 2
Overall response rate (ORR), complete response (CR), partial response (PR), stable disease
(SD) and progressive disease (PD) of patients treated with 177Lu-lilotomab satetraxetan

| | Best response | | | | | | | | |
|---|---|---|---|---|---|---|---|---|---|
| | Arm 1 | | | Arm 2 | | Arm 3 | Arm 4 | | Total |
| Activity | 10 | 15 | 20 | 10 | 15 | 15 | 15 | 20 | |
| (MBq/kg) | | | | | | | | | |
| n | 4 | 29 | 3 | 1 | 2 | 3 | 3 | 7 | 52 |
| ORR | 2 | 20 | 3 | 1 | 1 | 2 | 1 | 3 | 33 |
| (CR + PR) | (50%) | (69%) | (100%) | (100%) | (50%) | (67%) | (33%) | (43%) | (63%) |
| CR | 0 | 9 | 2 | 0 | 0 | 0 | 1 | 1 | 13 |
| | | (31%) | (67%) | | | | (33%) | (14%) | (25%) |
| PR | 2 | 11 | 1 | 1 | 1 | 2 | 0 | 2 | 20 |
| | (50%) | (38%) | (33%) | (100%) | (50%) | (67%) | | (29%) | (38%) |
| SD | 1 | 3 | 0 | 0 | 1 | 0 | 1 | 3 | 9 |
| | (25%) | (10%) | | | (50%) | | (33%) | (43%) | (17%) |
| PD | 1 | 4 | 0 | 0 | 0 | 1 | 1 | 1 | 10 |
| | (25%) | (17%) | | | | (33%) | (33%) | (14%) | (19%) |

Conclusion:

The hematological toxicity was reduced by pre-dosing with lilotomab.

Items

1. ¹⁷⁷Lu-lilotomab satetraxetan for use the treatment of Non-Hodgkin lymphoma, wherein ¹⁷⁷Lu-lilotomab satetraxetan is administered according to an administration pattern comprising:

a) predosing of 20-250 mg/m² lilotomab, followed by
b) 10-50 MBq/kg ¹⁷⁷Lu-lilotomab satetraxetan
to a person in need thereof.

2. ¹⁷⁷Lu-lilotomab satetraxetan for use in the treatment of Non-Hodgkin lymphoma according to item 1, wherein ¹⁷⁷Lu-lilotomab satetraxetan is administered at a concentration selected from the group consisting of 10, 12.5, 15, 17.5, 20, 25, 30, 35, 40, 45 and 50 MBq/kg.

3. ¹⁷⁷Lu-lilotomab satetraxetan for use in the treatment of Non-Hodgkin lymphoma according to any one of items 1-2, wherein ¹⁷⁷Lu-lilotomab satetraxetan is administered at a concentration 15 MBq/kg.

4. ¹⁷⁷Lu-lilotomab satetraxetan for use in the treatment of Non-Hodgkin lymphoma according to any one of items 1-3, wherein ¹⁷⁷Lu-lilotomab satetraxetan is administered at a concentration 17.5 MBq/kg.

5. ¹⁷⁷Lu-lilotomab satetraxetan for use in the treatment of Non-Hodgkin lymphoma according to any one of items 1-4, wherein ¹⁷⁷Lu-lilotomab satetraxetan is administered at a concentration 20 MBq/kg.

6. ¹⁷⁷Lu-lilotomab satetraxetan for use in the treatment of Non-Hodgkin lymphoma according to any one of items 1-5, wherein lilotomab is administered at a concentration selected from the group consisting of 20 mg/m², 40 mg/m², 50 mg/m², 60 mg/m², 75 mg/m², 100 mg/m², 125 mg/m², 150 mg/m², 200 mg/m², 250 mg/m², 20 mg/patient and 40 mg/patient.

7. ¹⁷⁷Lu-lilotomab satetraxetan for use in the treatment of Non-Hodgkin lymphoma according to any one of items 1-6, wherein lilotomab is administered at 20 mg/m2.

8. ¹⁷⁷Lu-lilotomab satetraxetan for use in the treatment of Non-Hodgkin lymphoma according to any one of items 1-7, wherein lilotomab is administered at 40 mg/m².

9. ¹⁷⁷Lu-lilotomab satetraxetan for use in the treatment of Non-Hodgkin lymphoma according to any one of items 1-8, wherein lilotomab is administered at 60 mg/m².

10. ¹⁷⁷Lu-lilotomab satetraxetan for use in the treatment of Non-Hodgkin lymphoma according to any one of items 1-8, wherein lilotomab is administered at 100 mg/m².

11. ¹⁷⁷Lu-lilotomab satetraxetan for use in the treatment of Non-Hodgkin lymphoma according to any one of items 1-9, wherein lilotomab is administered at 20 mg/patient.

12. ¹⁷⁷Lu-lilotomab satetraxetan for use in the treatment of Non-Hodgkin lymphoma according to any one of items 1-9, wherein lilotomab is administered at 40 mg/patient.

13. ¹⁷⁷Lu-lilotomab satetraxetan for use in the treatment of Non-Hodgkin lymphoma according to any one of items 1-12, wherein the predosing of lilotomab is done less than 24 hours, such as within 4 hours before administration of ¹⁷⁷Lu-lilotomab satetraxetan.

14. ¹⁷⁷Lu-lilotomab satetraxetan for use in the treatment of Non-Hodgkin lymphoma according to any one of items 1-13, further comprising a pretreatment step before step a), wherein the pretreatment step comprises pretreatment with one, two, three or more administrations of 375 mg/m² rituximab.

15. ¹⁷⁷Lu-lilotomab satetraxetan for use in the treatment of Non-Hodgkin lymphoma according to item 14, wherein 375 mg/m² rituximab is administered at 28 and 21 days before administration of ¹⁷⁷Lu-lilotomab satetraxetan.

16. ¹⁷⁷Lu-lilotomab satetraxetan for use in the treatment of Non-Hodgkin lymphoma according to item 14, wherein 375 mg/m² rituximab is administered at 14 days before administration of ¹⁷⁷Lu-lilotomab satetraxetan.

17. ¹⁷⁷Lu-lilotomab satetraxetan for use in the treatment of Non-Hodgkin lymphoma according to items 14 and 16, wherein 375 mg/m² rituximab is infused at 14 days before and again less than 4 hours before administration of ¹⁷⁷Lu-lilotomab satetraxetan.

18. ¹⁷⁷Lu-lilotomab satetraxetan for use in the treatment of Non-Hodgkin lymphoma according to any one of items 1-17, wherein the lymphoma is a subtype selected from the group consisting of follicular grade I-IIIA, marginal zone, small lymphocytic, lymphoplasmacytic, AML, CLL, BLBCL, AML with 11Q23/MLL translocation, and mantle cell.

19. ¹⁷⁷Lu-lilotomab satetraxetan for use in the treatment of Non-Hodgkin lymphoma according to any one of items 1-12, wherein the patient is relapsing after treatment with rituximab.

20. Lilotomab for use in the treatment of Non-Hodgkin lymphoma wherein lilotomab is administered according to an administration pattern comprising:

a) predosing of 20-250 mg/m² lilotomab, followed by
b) 10-50 MBq/kg ¹⁷⁷Lu-lilotomab satetraxetan
to a person in need thereof.

21. Lilotomab for use in the treatment of Non-Hodgkin lymphoma wherein lilotomab is administered according to an administration pattern comprising:

a) predosing of 20-100 mg/m² lilotomab, followed by
b) 10-20 MBq/kg ¹⁷⁷Lu-lilotomab satetraxetan
to a person in need thereof.

22. Lilotomab for use in the treatment of Non-Hodgkin lymphoma wherein lilotomab is administered according to item 20-21, wherein ¹⁷⁷Lu-lilotomab satetraxetan is administered at a concentration 10 MBq/kg.

23. Lilotomab for use in the treatment of Non-Hodgkin lymphoma wherein lilotomab is administered according to item 20-21, wherein ¹⁷⁷Lu-lilotomab satetraxetan is administered at a concentration 15 MBq/kg.

24. Lilotomab for use in the treatment of Non-Hodgkin lymphoma wherein lilotomab is administered according to items 20-20, wherein ¹⁷⁷Lu-lilotomab satetraxetan is administered at a concentration 17.5 MBq/kg.

25. Lilotomab for use in the treatment of Non-Hodgkin lymphoma wherein lilotomab is administered according to items 20-21, wherein ¹⁷⁷Lu-lilotomab satetraxetan is 3 administered at a concentration 20 MBq/kg.

26. Lilotomab for use in the treatment of Non-Hodgkin lymphoma wherein lilotomab is administered according to items 20-21, wherein lilotomab is administered at 20 mg/m².

27. Lilotomab for use in the treatment of Non-Hodgkin lymphoma wherein lilotomab is administered according to items 20-21, wherein lilotomab is administered at 40 mg/m².

28. Lilotomab for use in the treatment of Non-Hodgkin lymphoma wherein lilotomab is administered according to items 20-21, wherein lilotomab is administered at 60 mg/m².

29. Lilotomab for use in the treatment of Non-Hodgkin lymphoma wherein lilotomab is administered according to items 20-21, wherein lilotomab is administered at 100 mg/m².

30. Lilotomab for use in the treatment of Non-Hodgkin lymphoma wherein lilotomab is administered according to items 20-21, wherein lilotomab is administered at 20 mg/patient.

31. Lilotomab for use in the treatment of Non-Hodgkin lymphoma wherein lilotomab is administered according to items 20-21, wherein lilotomab is administered at 40 mg/patient.

31. Lilotomab for use in the treatment of Non-Hodgkin lymphoma wherein lilotomab is administered according to items 20-21, wherein lilotomab is administered at 60 mg/kg.

31. Lilotomab for use in the treatment of Non-Hodgkin lymphoma wherein lilotomab is administered according to items 20-21, wherein lilotomab is administered at 100 mg/kg.

33. Lilotomab for use in the treatment of Non-Hodgkin lymphoma wherein lilotomab is administered according to an administration pattern comprising:

a) predosing of 40 mg/m² lilotomab, followed by
b) 15 MBq/kg ¹⁷⁷Lu-lilotomab satetraxetan
to a person in need thereof.

34. Lilotomab for use in the treatment of Non-Hodgkin lymphoma wherein lilotomab is administered according to an administration pattern comprising:

a) predosing of 40 mg/patient lilotomab, followed by
b) 15 MBq/kg ¹⁷⁷Lu-lilotomab satetraxetan
to a person in need thereof.

35. Lilotomab for use in the treatment of Non-Hodgkin lymphoma wherein lilotomab is administered according to an administration pattern comprising:

a) predosing of 100 mg/m² lilotomab, followed by
b) 15 MBq/kg ¹⁷⁷Lu-lilotomab satetraxetan
to a person in need thereof.

36. Lilotomab for use in the treatment of Non-Hodgkin lymphoma wherein lilotomab is administered according to an administration pattern comprising:

a) predosing of 60 mg/m² lilotomab, followed by
b) 10 MBq/kg ¹⁷⁷Lu-lilotomab satetraxetan
to a person in need thereof.

37. Lilotomab for use in the treatment of Non-Hodgkin lymphoma wherein lilotomab is administered according to an administration pattern comprising:

a) predosing of 60 mg/m² lilotomab, followed by
b) 15 MBq/kg ¹⁷⁷Lu-lilotomab satetraxetan
to a person in need thereof.

38. Lilotomab for use in the treatment of Non-Hodgkin lymphoma wherein lilotomab is administered according to an administration pattern comprising:

a) predosing of 60 mg/m² lilotomab, followed by
b) 20 MBq/kg ¹⁷⁷Lu-lilotomab satetraxetan
to a person in need thereof.

39. Lilotomab for use in the treatment of Non-Hodgkin lymphoma wherein lilotomab is administered according to an administration pattern comprising:

a) predosing of 20 mg/patient lilotomab, followed by
b) 15 MBq/kg ¹⁷⁷Lu-lilotomab satetraxetan
to a person in need thereof.

40. Lilotomab for use in the treatment of Non-Hodgkin lymphoma wherein lilotomab is administered according to an administration pattern comprising:

a) predosing of 20 mg/patient lilotomab, followed by
b) 20 MBq/kg ¹⁷⁷Lu-lilotomab satetraxetan
to a person in need thereof.

41. Lilotomab for use in the treatment of Non-Hodgkin lymphoma wherein lilotomab is administered according to an administration pattern comprising:

a) predosing of 20 mg/m² lilotomab, followed by
b) 15 MBq/kg ¹⁷⁷Lu-lilotomab satetraxetan
to a person in need thereof.

42. Lilotomab for use in the treatment of Non-Hodgkin lymphoma wherein lilotomab is administered according to an administration pattern comprising:

a) predosing of 100 mg/m² lilotomab, followed by
b) 20 MBq/kg ¹⁷⁷Lu-lilotomab satetraxetan
to a person in need thereof.

43. A combination of lilotomab and ¹⁷⁷Lu-lilotomab satetraxetan for use in the treatment of Non-Hodgkin lymphoma wherein lilotomab and ¹⁷⁷Lu-lilotomab satetraxetan is administered according to an administration pattern comprising:

a) predosing of 20-250 mg/m² lilotomab, followed by
b) 10-50 MBq/kg ¹⁷⁷Lu-lilotomab satetraxetan
to a person in need thereof.

44. A combination of lilotomab and ¹⁷⁷Lu-lilotomab satetraxetan for use according to item 43, wherein ¹⁷⁷Lu-lilotomab satetraxetan is administered at a concentration 10 MBq/kg.

45. A combination of lilotomab and ¹⁷⁷Lu-lilotomab satetraxetan for use according to item 43, wherein ¹⁷⁷Lu-lilotomab satetraxetan is administered at a concentration 15 MBq/kg.

46. A combination of lilotomab and ¹⁷⁷Lu-lilotomab satetraxetan for use according to item 43, wherein ¹⁷⁷Lu-lilotomab satetraxetan is administered at a concentration 17.5 MBq/kg.

47. A combination of lilotomab and ¹⁷⁷Lu-lilotomab satetraxetan for use according to item 43, wherein ¹⁷⁷Lu-lilotomab satetraxetan is administered at a concentration 20 MBq/kg.

48. A combination of lilotomab and ¹⁷⁷Lu-lilotomab satetraxetan for use according to item 36, wherein lilotomab is administered at 20 mg/m².

49. A combination of lilotomab and ¹⁷⁷Lu-lilotomab satetraxetan for use according to item 36, wherein lilotomab is administered at 40 mg/m².

50. A combination of lilotomab and ¹⁷⁷Lu-lilotomab satetraxetan for use according to item 36, wherein lilotomab is administered at 40 mg/patient.

51. A combination of lilotomab and ¹⁷⁷Lu-lilotomab satetraxetan for use according to item 36, wherein lilotomab is administered at 100 mg/m2.

52. A combination of lilotomab and ¹⁷⁷Lu-lilotomab satetraxetan for use according to item 36, wherein lilotomab is administered at 20 mg/patient.

53. A combination of lilotomab and ¹⁷⁷Lu-lilotomab satetraxetan for use in the treatment of Non-Hodgkin lymphoma wherein lilotomab and ¹⁷⁷Lu-lilotomab satetraxetan is administered according to an administration pattern comprising:

a) predosing of 40 mg/m² lilotomab, followed by
b) 15 MBq/kg ¹⁷⁷Lu-lilotomab satetraxetan
to a person in need thereof.

54. A combination of lilotomab and ¹⁷⁷Lu-lilotomab satetraxetan for use in the treatment of Non-Hodgkin lymphoma wherein lilotomab and ¹⁷⁷Lu-lilotomab satetraxetan is administered according to an administration pattern comprising:

a) predosing of 40 mg/patient lilotomab, followed by
b) 15 MBq/kg ¹⁷⁷Lu-lilotomab satetraxetan
to a person in need thereof.

55. A combination of lilotomab and ¹⁷⁷Lu-lilotomab satetraxetan for use in the treatment of Non-Hodgkin lymphoma wherein lilotomab and ¹⁷⁷Lu-lilotomab satetraxetan is administered according to an administration pattern comprising:

a) predosing of 100 mg/m² lilotomab, followed by
b) 15 MBq/kg ¹⁷⁷Lu-lilotomab satetraxetan
to a person in need thereof.

56. A combination of lilotomab and ¹⁷⁷Lu-lilotomab satetraxetan for use in the treatment of Non-Hodgkin lymphoma wherein lilotomab and ¹⁷⁷Lu-lilotomab satetraxetan is administered according to an administration pattern comprising:

a) predosing of 100 mg/m² lilotomab, followed by
b) 20 MBq/kg ¹⁷⁷Lu-lilotomab satetraxetan
to a person in need thereof.

57. Lilotomab for use in the reduction of haematological toxicity due to the administration of ¹⁷⁷Lu-lilotomab satetraxetan, wherein lilotomab is administered before ¹⁷⁷Lu-lilotomab satetraxetan in a dose of 20-250 mg/m².

58. Lilotomab for use in the reduction of haematological toxicity due to the administration of ¹⁷⁷Lu-lilotomab satetraxetan according to item 57, wherein lilotomab is administered before ¹⁷⁷Lu-lilotomab satetraxetan in a dose of 40 mg/m².

59. Lilotomab for use in the reduction of haematological toxicity due to the administration of ¹⁷⁷Lu-lilotomab satetraxetan according to item 57, wherein lilotomab is administered before ¹⁷⁷Lu-lilotomab satetraxetan in a dose of 40 mg/patient.

60. Lilotomab for use in the reduction of haematological toxicity due to the administration of ¹⁷⁷Lu-lilotomab satetraxetan according to item 57, wherein lilotomab is administered before ¹⁷⁷Lu-lilotomab satetraxetan in a dose of 100 mg/m².

61. Lilotomab for use in the reduction of haematological toxicity due to the administration of ¹⁷⁷Lu-lilotomab satetraxetan according to item 57, wherein lilotomab is administered before ¹⁷⁷Lu-lilotomab satetraxetan in a dose of 60 mg/m².

62. Lilotomab for use in the reduction of haematological toxicity due to the administration of ¹⁷⁷Lu-lilotomab satetraxetan according to items 57-61, wherein ¹⁷⁷Lu-lilotomab satetraxetan is administered in a dose of 15 MBq/kg.

63. Lilotomab for use in the reduction of haematological toxicity due to the administration of ¹⁷⁷Lu-lilotomab satetraxetan according to items 57-61, wherein ¹⁷⁷Lu-lilotomab satetraxetan is administered in a dose of 17.5 MBq/kg.

64. Lilotomab for use in the reduction of haematological toxicity due to the administration of ¹⁷⁷Lu-lilotomab satetraxetan according to items 57-61, wherein ¹⁷⁷Lu-lilotomab satetraxetan is administered in a dose of 20 MBq/kg.

65. Lilotomab for use in the reduction of haematological toxicity due to the administration of 15 MBq/kg ¹⁷⁷Lu-lilotomab satetraxetan, wherein lilotomab is administered before ¹⁷⁷Lu-lilotomab satetraxetan in a dose of 40 mg/m².

66. Lilotomab for use in the reduction of haematological toxicity due to the administration of 15 MBq/kg ¹⁷⁷Lu-lilotomab satetraxetan, wherein lilotomab is administered before ¹⁷⁷Lu-lilotomab satetraxetan in a dose of 40 mg/patient.

67. Lilotomab for use in the reduction of haematological toxicity due to the administration of 15 MBq/kg ¹⁷⁷Lu-lilotomab satetraxetan, wherein lilotomab is administered before ¹⁷⁷Lu-lilotomab satetraxetan in a dose of 100 mg/m².

68. Lilotomab for use in the reduction of haematological toxicity due to the administration of 20 MBq/kg ¹⁷⁷Lu-lilotomab satetraxetan, wherein lilotomab is administered before ¹⁷⁷Lu-lilotomab satetraxetan in a dose of 100 mg/m².

69. Lilotomab for use in the reduction of haematological toxicity due to the administration of 20 MBq/kg ¹⁷⁷Lu-lilotomab satetraxetan, wherein lilotomab is administered before ¹⁷⁷Lu-lilotomab satetraxetan in a dose of 40 mg/patient.

70. Lilotomab for use in the reduction of haematological toxicity due to the administration of 17.5 MBq/kg ¹⁷⁷Lu-lilotomab satetraxetan, wherein lilotomab is administered before ¹⁷⁷Lu-lilotomab satetraxetan in a dose of 40 mg/patient.

71. Lilotomab for use in the reduction of haematological toxicity due to the administration of 15 MBq/kg ¹⁷⁷Lu-lilotomab satetraxetan, wherein lilotomab is administered before ¹⁷⁷Lu-lilotomab satetraxetan in a dose of 40 mg/patient.

72. Lilotomab for use in the reduction of haematological toxicity due to the administration of 15 MBq/kg ¹⁷⁷Lu-lilotomab satetraxetan, wherein lilotomab is administered before ¹⁷⁷Lu-lilotomab satetraxetan in a dose of 100 mg/m².

73. Lilotomab for use in the reduction of haematological toxicity due to the administration of 17.5 MBq/kg ¹⁷⁷Lu-lilotomab satetraxetan, wherein lilotomab is administered before ¹⁷⁷Lu-lilotomab satetraxetan in a dose of 100 mg/m2.

74. Lilotomab for use in the reduction of haematological toxicity due to the administration of 20 MBq/kg ¹⁷⁷Lu-lilotomab satetraxetan, wherein lilotomab is administered before ¹⁷⁷Lu-lilotomab satetraxetan in a dose of 100 mg/m².

75. Lilotomab for use in the reduction of haematological toxicity due to the administration of 10 MBq/kg ¹⁷⁷Lu-lilotomab satetraxetan, wherein lilotomab is administered before ¹⁷⁷Lu-lilotomab satetraxetan in a dose of 60 mg/m².

76. Lilotomab for use in the reduction of haematological toxicity due to the administration of 15 MBq/kg ¹⁷⁷Lu-lilotomab satetraxetan, wherein lilotomab is administered before ¹⁷⁷Lu-lilotomab satetraxetan in a dose of 60 mg/m².

77. Lilotomab for use in the reduction of haematological toxicity due to the administration of 20 MBq/kg ¹⁷⁷Lu-lilotomab satetraxetan, wherein lilotomab is administered before ¹⁷⁷Lu-lilotomab satetraxetan in a dose of 60 mg/m².

78. A method of treating Non-Hodgkin lymphoma comprising administration of ¹⁷⁷Lu-lilotomab satetraxetan in an administration pattern comprising:

a) predosing of 20-250 mg/m² lilotomab, followed by
b) 10-50 MBq/kg ¹⁷⁷Lu-lilotomab satetraxetan
to a person in need thereof.

79. A method of treating Non-Hodgkin lymphoma comprising administration of ¹⁷⁷Lu-lilotomab satetraxetan in an administration pattern comprising:

a) predosing of 20-500 mg/m² lilotomab, followed by
b) 10-50 MBq/kg ¹⁷⁷Lu-lilotomab satetraxetan
to a person in need thereof.

80. A method of treating Non-Hodgkin lymphoma comprising administration of ¹⁷⁷Lu-lilotomab satetraxetan in an administration pattern comprising:

a) predosing of 40 mg/m² lilotomab, followed by
b) 15 MBq/kg ¹⁷⁷Lu-lilotomab satetraxetan
to a person in need thereof.

81. A method of treating Non-Hodgkin lymphoma comprising administration of ¹⁷⁷Lu-lilotomab satetraxetan in an administration pattern comprising:

a) predosing of 40 mg/patient lilotomab, followed by
b) 15 MBq/kg ¹⁷⁷Lu-lilotomab satetraxetan
to a person in need thereof.

80. A method of treating Non-Hodgkin lymphoma comprising administration of ¹⁷⁷Lu-lilotomab satetraxetan in an administration pattern comprising:

a) predosing of 100 mg/m² lilotomab, followed by
b) 15 MBq/kg ¹⁷⁷Lu-lilotomab satetraxetan
to a person in need thereof.

83. A method of treating Non-Hodgkin lymphoma comprising administration of ¹⁷⁷Lu-lilotomab satetraxetan in an administration pattern comprising:

a) predosing of 100 mg/m² lilotomab, followed by
b) 20 MBq/kg ¹⁷⁷Lu-lilotomab satetraxetan
to a person in need thereof.

84. ¹⁷⁷Lu-lilotomab satetraxetan for use the treatment of Non-Hodgkin lymphoma, wherein ¹⁷⁷Lu-lilotomab satetraxetan is administered according to an administration pattern comprising:

0) pretreatment with 375 mg/m² rituximab administered at 14 days before administration of ¹⁷⁷Lu-lilotomab satetraxetan,
a) predosing of 100 mg/m² lilotomab less than 4 hours before administration of ¹⁷⁷Lu-lilotomab satetraxetan, followed by
b) 15 MBq/kg ¹⁷⁷Lu-lilotomab satetraxetan
to a person in need thereof.

85. ¹⁷⁷Lu-lilotomab satetraxetan for use the treatment of Non-Hodgkin lymphoma, wherein ¹⁷⁷Lu-lilotomab satetraxetan is administered according to an administration pattern comprising:

0) pretreatment with 375 mg/m² rituximab administered at 14 days before administration of ¹⁷⁷Lu-lilotomab satetraxetan,
a) predosing of 100 mg/m² lilotomab less than 4 hours before administration of ¹⁷⁷Lu-lilotomab satetraxetan, followed by
b) 20 MBq/kg ¹⁷⁷Lu-lilotomab satetraxetan
to a person in need thereof.

86. ¹⁷⁷Lu-lilotomab satetraxetan for use the treatment of Non-Hodgkin lymphoma, wherein ¹⁷⁷Lu-lilotomab satetraxetan is administered according to an administration pattern comprising:

0) pretreatment with 375 mg/m² rituximab administered at 14 days before administration of ¹⁷⁷Lu-lilotomab satetraxetan,
a) predosing of 100 mg/m² lilotomab less than 4 hours before administration of ¹⁷⁷Lu-lilotomab satetraxetan, followed by
b) 25 MBq/kg ¹⁷⁷Lu-lilotomab satetraxetan
to a person in need thereof.

87. ¹⁷⁷Lu-lilotomab satetraxetan for use the treatment of Non-Hodgkin lymphoma, wherein ¹⁷⁷Lu-lilotomab satetraxetan is administered according to an administration pattern comprising:

0) pretreatment with 375 mg/m² rituximab administered at 14 days before administration of ¹⁷⁷Lu-lilotomab satetraxetan,
a) predosing of 100 mg/m² lilotomab less than 4 hours before administration of ¹⁷⁷Lu-lilotomab satetraxetan, followed by
b) 30 MBq/kg ¹⁷⁷Lu-lilotomab satetraxetan
to a person in need thereof.

88. ¹⁷⁷Lu-lilotomab satetraxetan for use the treatment of Non-Hodgkin lymphoma, wherein ¹⁷⁷Lu-lilotomab satetraxetan is administered according to an administration pattern comprising:

0) pretreatment with 375 mg/m² rituximab administered at 14 days before administration of ¹⁷⁷Lu-lilotomab satetraxetan,
a) predosing of 60 mg/m² lilotomab less than 4 hours before administration of ¹⁷⁷Lu-lilotomab satetraxetan, followed by
b) 15 MBq/kg ¹⁷⁷Lu-lilotomab satetraxetan
to a person in need thereof.

89. ¹⁷⁷Lu-lilotomab satetraxetan for use the treatment of Non-Hodgkin lymphoma, wherein ¹⁷⁷Lu-lilotomab satetraxetan is administered according to an administration pattern comprising:

0) pretreatment with 375 mg/m² rituximab administered at 14 days before administration of ¹⁷⁷Lu-lilotomab satetraxetan,
a) predosing of 60 mg/m² lilotomab less than 4 hours before administration of ¹⁷⁷Lu-lilotomab satetraxetan, followed by
b) 20 MBq/kg ¹⁷⁷Lu-lilotomab satetraxetan
to a person in need thereof.

90. ¹⁷⁷Lu-lilotomab satetraxetan for use the treatment of Non-Hodgkin lymphoma, wherein ¹⁷⁷Lu-lilotomab satetraxetan is administered according to an administration pattern comprising:

0) pretreatment with 375 mg/m² rituximab administered at 14 days before administration of ¹⁷⁷Lu-lilotomab satetraxetan,
a) predosing of 40 mg/m² lilotomab less than 4 hours before administration of ¹⁷⁷Lu-lilotomab satetraxetan, followed by
b) 15 MBq/kg ¹⁷⁷Lu-lilotomab satetraxetan
to a person in need thereof.

91. ¹⁷⁷Lu-lilotomab satetraxetan for use the treatment of Non-Hodgkin lymphoma, wherein ¹⁷⁷Lu-lilotomab satetraxetan is administered according to an administration pattern comprising:

0) pretreatment with 375 mg/m² rituximab administered at 14 days before administration of ¹⁷⁷Lu-lilotomab satetraxetan,
a) predosing of 40 mg/patient lilotomab less than 4 hours before administration of ¹⁷⁷Lu-lilotomab satetraxetan, followed by
b) 15 MBq/kg ¹⁷⁷Lu-lilotomab satetraxetan
to a person in need thereof.

92. ¹⁷⁷Lu-lilotomab satetraxetan for use the treatment of Non-Hodgkin lymphoma, wherein ¹⁷⁷Lu-lilotomab satetraxetan is administered according to an administration pattern comprising:

0) pretreatment with 375 mg/m² rituximab administered at 14 days before administration of ¹⁷⁷Lu-lilotomab satetraxetan,
a) predosing of 40 mg/patient lilotomab less than 4 hours before administration of ¹⁷⁷Lu-lilotomab satetraxetan, followed by
b) 20 MBq/kg ¹⁷⁷Lu-lilotomab satetraxetan
to a person in need thereof.

1. A method of inhibiting Non-Hodgkin lymphoma comprising administering 15-20 MB q/kg ¹⁷⁷Lu-lilotomab satetraxetan according to a clinically relevant administration pattern
to a person in need thereof.

2-28. (canceled)

29. The method according to claim 1, wherein the ¹⁷⁷Lu-lilotomab satetraxetan is administered to said person according to an administration pattern comprising:
a) predosing of 20-100 mg/m² lilotomab, followed by
b) 15-20 MBq/kg ¹⁷⁷Lu-lilotomab satetraxetan.

30. The method according to claim 1, wherein the ¹⁷⁷Lu-lilotomab satetraxetan is administered at a concentration of 15 MBq/kg.

31. The method according to claim 1, wherein the ¹⁷⁷Lu-lilotomab satetraxetan is administered at a concentration of 20 MBq/kg.

32. The method according to claim 29, wherein the lilotomab is administered at 100 mg/m².

33. The method according to claim 29, wherein the lilotomab is administered at 40 mg/patient.

34. The method according to claim 29, wherein the predosing of lilotomab is done less than 24 hours before administration of ¹⁷⁷Lu-lilotomab satetraxetan.

35. The method according to claim 1, further comprising a pretreatment step before step a), wherein the pretreatment step comprises pretreatment with one, two, three or more administrations of 375 mg/m² rituximab.

36. The method according to claim 35, wherein 375 mg/m² rituximab is administered at 28 and 21 days before administration of the ¹⁷⁷Lu-lilotomab satetraxetan.

37. The method according to claim 36, wherein 375 mg/m² rituximab is administered at 14 days before administration of ¹⁷⁷Lu-lilotomab satetraxetan.

38. The method according to claim 1, wherein the lymphoma is a subtype selected from the group consisting of follicular grade I-IIIA, marginal zone, small lymphocytic, lymphoplasmacytic, AML, CLL, Diffuse large B-cell lymphoma, AML with 11Q23/MLL translocation, and mantle cell.

39. The method according to claim 1, wherein the person is relapsing after treatment with anti-CD20 antibody therapy or is refractory to anti-CD20 antibody therapy.

40. A method of inhibiting Non-Hodgkin lymphoma comprising administering ¹⁷⁷Lu-lilotomab satetraxetan according to an administration pattern comprising:
0) pretreatment with 375 mg/m² rituximab administered at 14 days before administration of ¹⁷⁷Lu-lilotomab satetraxetan,
a) predosing of 40 mg lilotomab less than 4 hours before administration of ¹⁷⁷Lu-lilotomab satetraxetan, followed by
b) 15-20 MBq/kg ¹⁷⁷Lu-lilotomab satetraxetan
to a person in need thereof.

41. A method of inhibiting Non-Hodgkin lymphoma comprising administering ¹⁷⁷Lu-lilotomab satetraxetan according to an administration pattern comprising:
0) pretreatment with 375 mg/m² rituximab administered at 14 days before administration of ¹⁷⁷Lu-lilotomab satetraxetan,
a) predosing of 100 mg/m² lilotomab less than 4 hours before administration of ¹⁷⁷Lu-lilotomab satetraxetan, followed by
b) 15-20 MBq/kg ¹⁷⁷Lu-lilotomab satetraxetan
to a person in need thereof.
